(19) United States
(12) Patent Application Publication
(10) Pub. No.: US 2014/0073813 A1
(43) Pub. Date: Mar. 13, 2014

(54) Heat Integration of Carbonylation and Aldol Condensation Reaction Processes

(71) Applicant: Celanese International Corporation, Irving, TX (US)

(72) Inventors: Tianshu Pan, Houston, TX (US); Ronald D. Shaver, Houston, TX (US); G. Paull Torrence, League City, TX (US); Josefina T. Chapman, Houston, TX (US); Sean Mueller, Pasadena, TX (US)

(73) Assignee: Celanese International Corporation, Irving, TX (US)

(21) Appl. No.: 14/018,574

(22) Filed: Sep. 5, 2013

Related U.S. Application Data
(60) Provisional application No. 61/700,541, filed on Sep. 13, 2012.

(51) Int. Cl. C07C 51/347

(52) U.S. Cl. CPC C07C 51/347
USPC 562/519

(57) ABSTRACT

In one embodiment, the invention is to a process for producing an acrylic acid, comprising the step of reacting, in a carbonylation system, carbon monoxide with at least one reactant in a reaction medium under conditions effective to produce a crude alkanoic acid stream comprising alkanoic acid. Preferably, the reaction is an exothermic carbonylation reaction. The process further comprises the step of removing from the carbonylation system at least a portion of heat generated by the carbonylation reaction and transferring a portion of the heat to a heat transfer system that utilizes at least one steam condensate stream. The process further comprises the step of conveying at least a portion of the heat transferred to the heat transfer system of the condensation reaction zone and/or the condensation separation zone.

### CROSS REFERENCE TO RELATED APPLICATION

This application claims priority to U.S. Provisional Application No. 61/700,541, filed on Sep. 13, 2012, which is incorporated herein by reference.

### FIELD OF THE INVENTION

The present invention relates generally to the production of acrylate product. Specifically, the process relates to the integration of heat generated by the carbonylation process into the aldol condensation reaction process.

### BACKGROUND OF THE INVENTION

α,β-unsaturated acids, particularly acrylic acid and methacrylic acid, and the ester derivatives thereof are useful organic compounds in the chemical industry. These acids and esters are known to readily polymerize or co-polymerize to form homopolymers or copolymers. Often the polymerized acids are useful in applications such as superabsorbents, dispersants, flocculants, and thickeners. The polymerized ester derivatives are used in coatings (including latex paints), textiles, adhesives, plastics, fibers, and synthetic resins.

Because acrylic acid and its esters have long been valued commercially, many methods of production have been developed. One exemplary acrylic acid ester production process utilizes: (1) the reaction of acetylene with water and carbon monoxide; and/or (2) the reaction of an alcohol and carbon monoxide, in the presence of an acid, e.g., hydrochloric acid, and nickel tetracarbonyl, to yield a crude product comprising the acrylate ester as well as hydrogen and nickel chloride. Another conventional process involves the reaction of ketene (often obtained by the pyrolysis of acetone or acetic acid) with formaldehyde, which yields a crude product comprising acrylic acid and either water (when acetic acid is used as a pyrolysis reactant) or methane (when acetone is used as a pyrolysis reactant). These processes have become obsolete for economic, environmental, or other reasons.

More recent acrylic acid production processes have relied on the gas phase oxidation of propylene, via acrolein, to form acrylic acid. The reaction can be carried out in single- or two-step processes but the latter is favored because of higher yields. The oxidation of propylene produces acrolein, acrylic acid, acetaldehyde and carbon oxides. Acrylic acid from the primary oxidation can be recovered while the acrolein is fed to a second step to yield the crude acrylic acid product, which comprises acrylic acid, water, small amounts of acetic acid, as well as impurities such as furfural, acrolein, and propionic acid. Purification of the crude product may be carried out by azeotropic distillation. Although this process may show some improvement over earlier processes, this process suffers from production and/or separation inefficiencies. In addition, this oxidation reaction is highly exothermic and, as such, creates an explosion risk. As a result, more expensive reactor design and metallurgy are required. Also, the cost of propylene is often prohibitive.

The aldol condensation reaction of formaldehyde and acetic acid and/or carboxylic acid esters has been disclosed in literature. This reaction forms acrylic acid and is often conducted over a catalyst. For example, condensation catalysts consisting of mixed oxides of vanadium and phosphorus were investigated and described in M. Ai, J. Catal., 107, 201 (1987); M. Ai, J. Catal., 124, 293 (1990); M. Ai, Appl. Catal., 36, 221 (1988); and M. Ai, Shokubai, 29, 522 (1987). The acetic acid conversions in these reactions, however, may leave room for improvement. Although this reaction is disclosed, there has been little if any disclosure relating to: 1) the effects of reactant feed parameters on the aldol condensation crude product; or 2) separation schemes that may be employed to effectively provide purified acrylic acid from the aldol condensation crude product.

Some processes for producing acetic acid, which may be used in the aldol condensation reaction, are also disclosed. One example is a methanol carbonylation process. These processes typically yield a finished acetic acid product having less than 0.15 wt. % water, which is preferred for most acetic acid applications. To achieve this level of purity, however, significant separation resources must be employed.

U.S. Pat. No. 6,180,821 describes an integration process of acetic acid and/or vinyl acetate from ethylene, or ethane, using a first reaction zone with a catalyst active for the oxidation of ethylene to acetic acid and/or active for the oxidation of ethane to acetic acid, ethylene and carbon monoxide, and a second reaction zone containing catalyst active for the production of vinyl acetate. U.S. Pat. No. 7,465,823 describes an integrated process for the production of acetic acid and vinyl acetate monomers.

U.S. Pat. App. 2012/0071688 teaches a process for preparing acrylic acid from methanol and acetic acid in two separate reaction zones. In a first reaction zone, methanol is partially oxidized to formaldehyde in a heterogeneously catalyzed gas phase reaction to obtain a gas mixture that is typically further treated to provide a first product of formaldehyde/water solution. Excess amount of acetic acid is added to the first product to obtain a second product, which comprises unreacted acetic acid and formaldehyde. The formaldehyde and acetic acid is catalytically aldol condensed to form a product mixture including acrylic acid and unreacted acetic acid under heterogeneous catalysis. The unreacted acetic acid in the product mixture is removed and recycled into the production of acrylic acid.

Even in view of the references, the need remains for an acrylate product production process that integrates heat streams generated from the carbonylation process into the aldol condensation reaction process thus providing for efficiency improvements in the respective separation schemes.

### SUMMARY OF THE INVENTION

In a first embodiment, the present invention is directed to a process for producing acrylate product, e.g., acrylic acid. The process comprises the step of reacting, in a carbonylation system, carbon monoxide with at least one reactant in a reaction medium under conditions effective to product a crude alkanoic acid stream comprising alkanoic acid. Preferably, the reaction is an exothermic carbonylation reaction and the reaction medium comprises water, methyl iodide, and a first catalyst. The process further comprises the step of separating the crude alkanoic acid stream to form an alkanoic acid product stream comprising alkanoic acid and water. The process further comprises removing from the carbonylation system at least a portion of heat generated by the carbonylation reaction. The process further comprises the step of transferring at least a portion of the heat generated by the carbonylation reaction to a heat transfer system that utilizes at least one steam condensate stream to convey the generated heat. The process further comprises the step of contacting, in a condensation reaction zone, at least a portion of the alkanoic acid in the alkanoic acid product stream with an alkylenating agent in a reactor in the presence of a second catalyst under conditions effective to form a crude acrylate product stream comprising the acrylate product. The process further comprises conveying at least a portion of the at least one steam condensate stream to the condensation reaction zone. The process further comprises separating, in a condensation separation zone, the crude acrylate product stream to form an acrylate product stream and a water stream.

The process further comprises conveying a portion of the heat of the at least one steam condensate stream to a vaporizer or multiple vaporizers which are running in parallel and/or in series. The process further comprises vaporizing the alkanoic acid product stream and the alkylenating agent in the condensation reaction zone using the at least one steam condensate stream. Preferably, the at least one steam condensate stream has a temperature of at least 130° C.

The process further comprises employing a portion of the heat of the at least one steam condensate stream to preheat the recycle stream(s) fed to the reactor. The process further comprises conveying a portion of the heat of the at least one steam condensate stream to the condensation zone. The process further comprises returning at least a portion of the at least one steam condensate stream to the carbonylation system.

The condensation separation zone of the invention further comprises at least one column and at least a portion of the at least one steam condensate stream provides heat to the at least one column. In one embodiment, the at least one steam condensate stream is conveyed to a reboil stream of the at least one column of the condensation separation zone.

The process further comprises separating the crude alkanoic acid in a light ends column. The process further comprising withdrawing the alkanoic acid product stream as a sidedraw from the light ends column. In one embodiment, the alkanoic acid product stream comprises from 0.5 wt. % to 25 wt. % water.

The process further comprises separating at least a portion of the crude alkylate product stream to form an alklenating agent stream comprising at least 1 wt. % alkylenating agent and a product stream comprising at least 10 wt. % acrylate product.

### BRIEF DESCRIPTION OF DRAWINGS

The invention is described in detail below with reference to the appended drawings, wherein like numerals designate similar parts.

FIG. 1 is a diagram of an acetic acid and acrylic acid integrated production process in accordance with one embodiment of the present invention.

FIG. 2 is a schematic diagram of an exemplary integrated carbonylation and condensation process in accordance with one embodiment of the present invention.

FIG. 3 is a schematic diagram of an exemplary integrated carbonylation and condensation process in accordance with one embodiment of the present invention.

FIG. 4 is a schematic diagram of an exemplary integrated carbonylation and condensation process in accordance with one embodiment of the present invention.

FIG. 5 is a chart showing the hot stream and cold stream requirements for the carbonylation process.

FIG. 6 is a chart showing the hot stream and cold stream requirements for the aldol condensation reaction process.

FIG. 7 is a chart showing the integration of hot stream and cold stream requirements for the carbonylation and aldol condensation reaction processes.

### DETAILED DESCRIPTION OF THE INVENTION

Production of unsaturated carboxylic acids such as acrylic acid and methacrylic acid and the ester derivatives thereof via most conventional processes have been limited by economic and environmental constraints. In the interest of finding a new reaction path, the aldol condensation reaction of an alkanoic acid, e.g., acetic acid and an alkylenating agent, e.g., formaldehyde, has been investigated.

The present invention relates to a process for producing an acrylate product, e.g., acrylic acid, by the aldol condensation reaction of an alkanoic acid (provided via an alkanoic acid feed) and an alkylenating agent in the presence of a catalyst. The preheat of reaction feeds to condensation reaction temperature and separation processes for making the acrylate product are energy intensive. Typically, the preheat of reaction feeds to the temperature of condensation reaction of alkanoic acid and alkylenating agent requires about 3 to 10 mmbtu/tone acrylic acid. The separation process involves a number of distillation columns and the energy required to product glacial acrylic acid could be high. In the present invention, the alkanoic acid is made by an exothermic carbonylation reaction of methanol and carbon monoxide. Therefore, it is preferable that the heat generated by the exothermic carbonylation reaction be captured and used as a heat source for the condensation reaction and separation process of the acrylate product.

In accordance to one embodiment of the present invention, the heat generated by the carbonylation system may be transferred to a suitable heat transfer system. The heat transfer system may transfer the heat to the condensation reaction zone and/or separation zone. Depending on the specific configuration of the system, the heat may be conveyed to various locations of the condensation reaction zone and/or separation zone. For example, heat may be conveyed from the carbonylation reaction reactor to preheat and/or vaporize the condensation reaction feed streams of alkanoic acid and alkylenating agent prior to the condensation reaction. Heat may also be conveyed from the carbonylation reaction to preheat the recycled streams that are fed to the condensation reaction zone. In addition, the heat may be conveyed to various components in the condensation separation zone, such as to one or more distillation columns. Alternatively, the processes and systems described herein may be used to allocate portions of the heat transferred among more than one of the locations within the acrylate production process.

In one embodiment, the heat transfer system may utilize at least one steam condensate stream to convey the generated heat from the carbonylation reaction to the condensation system. Preferably, the at least one steam condensate stream may be directed to the condensation system, including the condensation reaction zone and the separation zone. Preferably, the at least one steam condensate stream may be direct to a vaporizer or a preheat of the condensation reaction zone, or one or more distillation columns of the condensation separation zone. In one embodiment, the at least one steam condensate stream provides heat to the vaporizer(s), which vaporize(s) the alkanoic acid, water, and/or alkylenating agent. In one embodiment, the at least one steam condensate stream provides heat to preheat the recycled streams that are fed to the reactor where the crude acrylate product is formed. In one embodiment, the at least one steam condensate stream provides heat to the one or more distillation columns, where acrylate product, e.g., acrylic acid, alkylenating agent, and water are separated. In one embodiment, the at least one steam condensate stream has a temperature of at least 120° C., e.g., at least 150° C. or at least 180° C. In one embodiment, the at least one steam condensate stream has a temperature from 180° C. to 200° C., e.g., from 150° C. to 180° C., or from 120° C. to 150° C. As a result of the use of at least one steam condensate stream to provide energy to the condensation system, less energy is demanded from outside sources which provide utility steam, utility fuels, and/or electricity. The heat from the carbonylation reaction provides at least a portion of the energy necessary for the condensation reaction zone and/or the condensation separation zone. Thus, by integrating the carbonylation system with the condensation system, the overall operating cost of producing acrylate product is reduced.

In one embodiment, the present invention is to a process for producing acrylic acid, methacrylic acid, and/or the salts and esters thereof. As used herein, acrylic acid, methacrylic acid, and/or the salts and esters thereof, collectively or individually, may be referred to as “acrylate products.” The use of the terms acrylic acid, methacrylic acid, or the salts and esters thereof, individually, does not exclude the other acrylate products, and the use of the term acrylate product does not require the presence of acrylic acid, methacrylic acid, and the salts and esters thereof.

In one embodiment, the process of the present invention comprises the step of reacting carbon monoxide with at least one reactant, e.g., methanol, in a reaction medium under condition effective to product a crude alkanoic acid stream. The carbonylation reaction can happen in either heterogeneous or homogeneous catalytic process. While the inventive process is illustrated for typical homogeneous catalytic process, same integration principles and analysis can be applied to the heterogeneous catalytic process In homogeneous catalytic process, the reaction medium further comprises water, methyl iodide and a first catalyst. The reaction between carbon monoxide and methanol is an exothermic carbonylation reaction and heat is generated from this reaction. An alkanoic acid product stream may be separated from the crude alkanoic acid stream and fed to a condensation reaction zone.

In one embodiment, the inventive process further comprises removing and transferring the heat generated by the carbonylation reaction to a heat transfer system. The heat transfer system utilizes at least one steam condensate stream to convey the generated heat to the condensation system, including the condensation reaction zone and the separation zone.

In one embodiment, the inventive process comprises the step of contacting at least a portion of the alkanoic acid in the alkanoic acid product stream with an alkylenating agent in a reactor in a condensation reaction zone in the presence of a second catalyst under conditions effective to form the crude acrylate product stream. The heat transfer system conveys at least one steam condensate stream to the condensation reaction zone. Preferably, the heat transfer system conveys heat to the vaporizer and/or the preheat of recycle streams. The heat conveyed by the steam condensate stream vaporizes the alkanoic acid and/or the alkylenating agent in the vaporizer. In one embodiment, the heat conveyed by the steam condensate stream provides heat to the preheat of recycle streams. The crude acrylate product stream is fed to the condensation separation zone to recover acrylate product.

In one embodiment, the inventive process comprises the step of conveying a portion of the heat of the at least one steam condensate stream to the condensation separation zone. The condensation separation zone may comprise a number of separation columns, such as distillation column, extraction column, or other separation components that requires heating. In one embodiment, a portion of the heat of the at least one steam condensate stream may be conveyed to one or more columns in the separation zone. In one embodiment, the at least one steam condensate stream is conveyed to a reboiled stream of one or more columns. The acrylate product may be recovered from the crude acrylate product stream.

In one embodiment, the steam condensate stream may be returned to the carbonylation system.

In one embodiment, the inventive process comprises the step of reacting at least a portion of the alkanoic acid, e.g., acetic acid, in the alkanoic acid feed stream with an alkylenating agent to form the crude acrylate product. The alkanoic acid feed stream comprises acetic acid and higher amounts of water, as compared to conventional acetic acid streams that are highly purified to remove water therefrom. In one embodiment, the alkanoic acid feed stream comprises water in amounts of up to 25 wt. %, e.g., up to 20 wt. % water, or up to 10 wt. % water. In terms of ranges the alkanoic acid feed stream may comprise from 0.15 wt. % to 25 wt. % water, e.g., from 0.2 wt. % to 20 wt. %, from 0.5 wt. % to 15 wt. %, or from 4 wt. % to 10 wt. %. In one embodiment, the alkanoic acid feed stream comprises water in an amount of at least 0.15 wt. %, e.g., at least 0.25 wt. %, at least 0.5 wt. %, or at least 2 wt. %. In some embodiments, the alkanoic acid feed stream may also comprise other carboxylic acids and anhydrides, as well as optionally acetaldehyde and/or acetone. In particular, the alkanoic acid feed stream may comprise methyl acetate and/or propanoic acid.

The crude acrylate product stream, in one embodiment, comprises acrylic acid and/or other acrylate products. The crude product stream of the present invention further comprises a significant portion of water and at least one alkylenating agent. In one embodiment, the crude product stream may comprise more water than would be produced from condensing glacial acetic acid and alkylenating agent. For example, the crude acrylate product stream may comprise more than 3 wt. % water, e.g., more than 10 wt. %, or more than 18 wt. %. In terms of ranges, the crude product stream may comprise from 3 wt. % to 80 wt. % water, e.g., from 10 wt. % to 70 wt. %, or from 18 wt. % to 60 wt. %. In terms of lower limits, the crude product stream may comprise at least 1 wt. % water, e.g., at least 5 wt. %, at least 10 wt. %, or at least 18 wt. %.

In one embodiment, the crude product stream may comprise at least 1 wt. % alkylenating agent, e.g., at least 3 wt. %, at least 5 wt. %, at least 7 wt. %, at least 10 wt. %, or at least 25 wt. %. In terms of ranges, the crude product stream may comprise from 1 wt. % to 50 wt. % alkylenating agent, e.g., from 1 wt. % to 45 wt. %, from 1 wt. % to 25 wt. %, from 1 wt. % to 10 wt. %, or from 5 wt. % to 10 wt. %. In terms of upper limits, the crude product stream may comprise less than 50 wt. % alkylenating agent, e.g., less than 45 wt. %, less than 25 wt. %, or less than 10 wt. %. Preferably, the at least one alkylenating agent is formaldehyde. The composition of the crude product stream is discussed in more detail below.

In one embodiment, the acetic acid may be produced by a carbonylation process. Conventional carbonylation processes yield a glacial acetic acid product comprising less than 1500 wppm water, e.g., less than 500 wppm, or less than 100 wppm. This product typically requires an energy intensive dehydrating step to achieve these low water levels. Embodiments of the present invention may, beneficially, eliminate the dehydrating step and/or allow the carbonylation process to run at improved operating conditions, e.g., lower energy requirements. Advantageously the present invention achieves an improvement in integration by allowing more water to be present in the acetic acid.

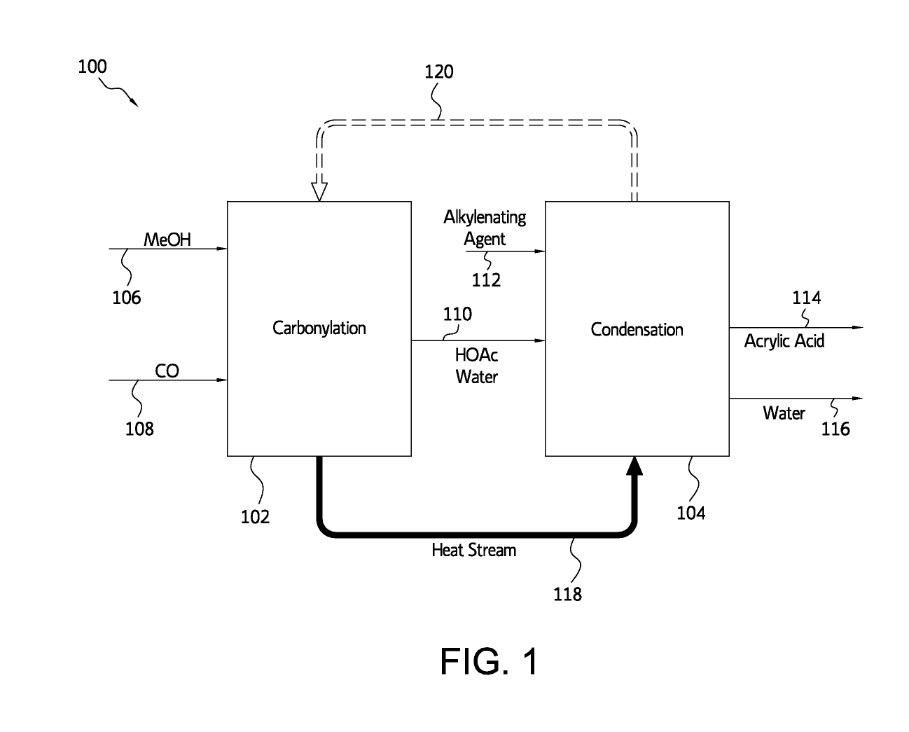

FIG. 1 is a diagram of integrated process 100 in accordance with the present invention. Process 100 comprises carbonylation system 102 and condensation system 104. Methanol feed 106 and carbon monoxide feed 108 are fed to carbonylation system 102. The methanol and the carbon monoxide are reacted in carbonylation system 102 to form a crude acetic acid product comprising acetic acid and water. A flasher (not shown in FIG. 1) may be used to remove residual catalyst from the crude product. Carbonylation system 102, in some embodiments, further comprises a purification zone comprising one or more distillation column (not shown in FIG. 1) to separate crude product into acetic acid product stream 110 comprising from 0.15 wt. % to 25 wt. % water.

Acetic acid product stream 110 is fed, more preferably directly fed, to condensation system 104. Some water may already be present in acetic acid product stream 110 and generally it is not necessary to further add water, e.g., to co-feed water. Thus, the water fed to condensation system 104 is provided by acetic acid product stream 110. Condensation system 104 also receives alkylenating agent feed 112. In some embodiments, alkylenating agent feed 112 may comprise water, which is co-fed to condensation system 104. In condensation system 104, the acetic acid in acetic acid product stream 110 is condensed with an alkylenating agent to form a crude acrylate product. The crude acrylate product may comprise acrylic acid and water and other compounds such as unreacted alkylenating agent, unreacted acetic acid, and components from side reactions. Also, the crude acrylate product may comprise additional gases that are introduced to improve the catalyst performances. Condensation system 104 may further comprise a condensation separation zone comprising one or more separation units, e.g. distillation columns, for recovering acrylic acid from the crude acrylate product. An acrylic acid product stream 114 may be recovered from condensation system 104. Also, the co-generated water 116 will be discharged from condensation system 104.

By productively delivering and utilizing heat energy generated in carbonylation process 102, substantial energy and cost savings may be achieved for the overall carbonylation/condensation system. During normal operation, the exothermic reaction of the methanol and carbon monoxide generates more heat than can be utilized in carbonylation process, e.g., in the separation zone thereof. Typically, this excess heat is removed without further application as an energy resource. For example, when the stream is withdrawn from the reactor and routed through a series of heat exchanger, a portion of the reaction heat is transferred to the cooling water and dissipated to the environment. After passing through the heat exchangers, the cooled stream may be returned to the reactor or be directed to other locations. The stream that is removed from and returned to the reactor is sometimes referred to as a reactor pump-around stream. These conventional processes have not yet effectively utilize the heat energy removed from the pump-around stream.

One embodiment of the present invention utilizes the excess heat energy that is typically wasted during the production of acetic acid. As shown in FIG. 1, one or more heat stream 118 may be conveyed from carbonylation system 102 to condensation system 104. In one embodiment, a heat transfer system (not shown) may be used to convey heat between the two systems. For example, one or more steam condensate stream may be used to absorb at least a portion of the reaction heat generated in carbonylation process 102. This heat may be used as energy resource in conjunction with the condensation system for the production and separation of acrylic acid. In one embodiment, one or more cooled steam condensate stream 120 may be returned to the carbonylation system 102 from the condensation system 104.

Carbonylation process 102 utilizes an exothermic reaction and, as such, achieves a high reaction temperature. For example, the carbonylation reaction between methanol and carbon monoxide generates a temperature greater than 115° C., e.g., greater than 150° C., or greater than 200° C. In one embodiment, the heat generated from the carbonylation reaction may be transferred to a heat transfer system that comprises one of more streams to generate at least one heat condensate stream 118 with temperature greater than 115° C., e.g., greater than 150° C., or greater than 200° C. The at least one steam condensate stream 118 may be used to provide heat to condensation system 104. For example, steam condensate stream 118 may be used to provide heat to the vaporizer to vaporize the feed streams. As another example, steam condensate stream 118 may be used to preheat the feed streams prior to feeding them to the vaporizer. In one embodiment, steam condensate stream 118 may be used to provide heat for one or more distillation columns for the purification of the crude acrylate product stream.

The process of the present invention may be used in any condensation process for producing acrylate products, e.g., acrylic acid. The materials, catalysts, reaction conditions, and separation processes that may be used in the integrated processes of the present invention are described further below.

### Raw Materials

Acetic acid is produced as an intermediate product in the present invention. The acetic acid may be derived from any suitable source including natural gas, petroleum, coal, biomass, and so forth. In some embodiments, the acetic acid may be produced via methanol carbonylation as described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, the entire disclosures of which are incorporated herein by reference.

As petroleum and natural gas prices fluctuate becoming either more or less expensive, methods for producing acetic acid and intermediates such as methanol and carbon monoxide from alternate carbon sources have drawn increasing interest. In particular, when petroleum is relatively expensive, it may become advantageous to produce acetic acid from synthesis gas (“syngas”) that is derived from more available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid.

In some embodiments, some or all of the raw materials for the above-described carbonylation and condensation integration processes may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. The syngas, in turn, may be derived from variety of carbon sources. The carbon source, for example, may be selected from the group consisting of natural gas, oil, petroleum, coal, biomass, and combinations thereof. Syngas may also be obtained from bio-derived methane gas, such as bio-derived methane gas produced by landfills or agricultural waste.

In another embodiment, the carbonylation-formed acetic acid used in the condensation system may be supplemented with acetic acid formed from the fermentation of biomass. The fermentation process preferably utilizes an acetogenic process or a homoacetogenic microorganism to ferment sugars to acetic acid producing little, if any, carbon dioxide as a by-product. The carbon efficiency for the fermentation process preferably is greater than 70%, greater than 80% or greater than 90% as compared to conventional yeast processing, which typically has a carbon efficiency of about 67%. Optionally, the microorganism employed in the fermentation process is of a genus selected from the group consisting of Clostridium, Lactobacillus, Moorella, Thermoanaerobacter, Propionibacterium, Propionispera, Anaerobiospirillum, and Bacteriodes, and in particular, species selected from the group consisting of Clostridium formicoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteriodes amylophilus and Bacteriodes ruminicola. Exemplary fermentation processes for forming acetic acid are disclosed in U.S. Pat. Nos. 6,509,180; 6,927,048; 7,074,603; 7,507,562; 7,351,559; 7,601,865; 7,682,812; and 7,888,082, the entireties of which are incorporated herein by reference. See also U.S. Pub. Nos. 2008/0193989 and 2009/0281354, the entireties of which are incorporated herein by reference.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. See, e.g., U.S. Pat. No. 7,884,253, the entirety of which is incorporated herein by reference. Another biomass source is black liquor, a thick, dark liquid that is a byproduct of the Kraft process for transforming wood into pulp, which is then dried to make paper. Black liquor is an aqueous solution of lignin residues, hemicellulose, and inorganic chemicals.

U.S. Pat. No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form syngas. The syngas is converted to methanol which may be carbonylated to acetic acid.

The acetic acid fed to the condensation zone may also comprise other carboxylic acids and anhydrides, as well as acetaldehyde and acetone. Preferably, a suitable acetic acid feed stream comprises one or more of the compounds selected from the group consisting of acetic acid, acetic anhydride, acetaldehyde, ethyl acetate, and mixtures thereof. In some embodiments, the presence of carboxylic acids, such as propanoic acid or its anhydride, may be beneficial in producing propanol. In some embodiments, water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the condensation zone of the present invention without the need for condensing the acetic acid and light ends or removing water, saving overall processing costs.

As used herein, “alkylenating agent” means an aldehyde or precursor to an aldehyde suitable for reacting with the alkanoic acid, e.g., acetic acid, to form an unsaturated acid, e.g., acrylic acid, or an alkyl acrylate. In preferred embodiments, the alkylenating agent comprises a methylenating agent such as formaldehyde, which preferably is capable of adding a methylene group (═CH₂) to the organic acid. Other alkylenating agents may include, for example, acetaldehyde, propanal, butanal, aryl aldehydes, benzyl aldehydes, alcohols, and combinations thereof. This listing is not exclusive and is not meant to limit the scope of the invention. In one embodiment, an alcohol may serve as a source of the alkylenating agent. For example, the alcohol may be reacted in situ to form the alkylenating agent, e.g., the aldehyde.

The alkylenating agent, e.g., formaldehyde, may be derived from any suitable source. Exemplary sources may include, for example, aqueous formaldehyde solutions, anhydrous formaldehyde derived from a formaldehyde drying procedure, trioxane, diether of methylene glycol, and paraformaldehyde. In a preferred embodiment, the formaldehyde is produced via a methanol oxidation process, which reacts methanol and oxygen to yield the formaldehyde.

In other embodiments, the alkylenating agent is a compound that is a source of formaldehyde. Where forms of formaldehyde that are not as freely or weakly complexed are used, the formaldehyde will form in situ in the condensation reactor or in a separate reactor prior to the condensation reactor. Thus for example, trioxane may be decomposed over an inert material or in an empty tube at temperatures over 350° C. or over an acid catalyst at over 100° C. to form the formaldehyde.

In one embodiment, the alkylenating agent corresponds to Formula I.

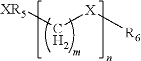

In this formula, R₅ and R₆ may be independently selected from C₁-C₁₂ hydrocarbons, preferably, C₁-C₁₂ alkyl, alkenyl or aryl, or hydrogen. Preferably, R₅ and R₆ are independently C₁-C₆ alkyl or hydrogen, with methyl and/or hydrogen being most preferred. X may be either oxygen or sulfur, preferably oxygen; and n is an integer from 1 to 10, preferably 1 to 3. In some embodiments, m is 1 or 2, preferably 1.

In one embodiment, the compound of formula I may be the product of an equilibrium reaction between formaldehyde and methanol in the presence of water. In such a case, the compound of formula I may be a suitable formaldehyde source. In one embodiment, the formaldehyde source includes any equilibrium composition. Examples of formaldehyde sources include but are not restricted to methylal (1,1 dimethoxymethane); polyoxymethylenes —(CH₂—O)ᵢ— wherein i is from 1 to 100; formalin; and other equilibrium compositions such as a mixture of formaldehyde, methanol, and methyl propionate. In one embodiment, the source of formaldehyde is selected from the group consisting of 1,1 dimethoxymethane; higher formals of formaldehyde and methanol; and CH₃—O—(CH₂—O)ᵢ—CH₃ where i is 2.

The alkylenating agent may be used with or without an organic or inorganic solvent.

The term “formalin,” refers to a mixture of formaldehyde, methanol, and water. In one embodiment, formalin comprises from 25 wt. % to 65 wt. % formaldehyde; from 0.01 wt. % to 25 wt. % methanol; and from 25 wt. % to 70 wt. % water. In cases where a mixture of formaldehyde, methanol, and methyl propionate is used, the mixture comprises less than 10 wt. % water, e.g., less than 5 wt. % or less than 1 wt. %.

### Carbonylation Reaction

In the carbonylation process, methanol is reacted with carbon monoxide in the presence of a carbonylation reactor under conditions effective to form acetic acid. Although carbonylation may be a preferred acetic acid production method, other suitable methods may be employed, e.g., in combination with carbonylation. In a preferred embodiment that employs carbonylation, the carbonylation system comprises a reaction zone, which includes a reactor, a flasher and optionally a reactor recovery unit. In one embodiment, carbon monoxide is reacted with methanol in a suitable reactor, e.g., a continuous stirred tank reactor (“CSTR”) or a bubble column reactor. The carbonylation of methanol, or another carbonylatable reactant, including, but not limited to, methyl acetate, methyl formate, dimethyl ether, or mixtures thereof, to acetic acid preferably occurs in the presence of a Group VIII metal catalyst, such as rhodium, and a halogen-containing catalyst promoter. Preferably, the carbonylation process is a low water, catalyzed, e.g., rhodium-catalyzed, carbonylation of methanol to acetic acid, as exemplified in U.S. Pat. No. 5,001,259, which is hereby incorporated by reference.

Without being bound by theory, the rhodium component of the catalyst system is believed to be present in the form of a coordination compound of rhodium with a halogen component providing at least one of the ligands of such coordination compound. In addition to the coordination of rhodium and halogen, it is also believed that carbon monoxide will coordinate with rhodium. The rhodium component of the catalyst system may be provided by introducing into the reaction zone rhodium in the form of rhodium metal, rhodium salts such as the oxides, acetates, iodides, carbonates, hydroxides, chlorides, etc., or other compounds that result in the formation of a coordination compound of rhodium in the reaction environment.

Suitable catalysts include Group VIII catalysts, e.g., rhodium and/or iridium catalysts. When a rhodium catalyst is utilized, the rhodium catalyst may be added in any suitable form such that the active rhodium catalyst is a carbonyl iodide complex. Exemplary rhodium catalysts are described in Michael Gauβ, et al., Applied Homogeneous Catalysis with Organometallic Compounds: A Comprehensive Handbook in Two Volume, Chapter 2.1, p. 27-200, (1^{st} ed., 1996). Iodide salts optionally maintained in the reaction mixtures of the processes described herein may be in the form of a soluble salt of an alkali metal or alkaline earth metal or a quaternary ammonium or phosphonium salt. In certain embodiments, a catalyst co-promoter comprising lithium iodide, lithium acetate, or mixtures thereof may be employed. The salt co-promoter may be added as a non-iodide salt that will generate an iodide salt. The iodide catalyst stabilizer may be introduced directly into the reaction system. Alternatively, the iodide salt may be generated in-situ since under the operating conditions of the reaction system, a wide range of non-iodide salt precursors will react with methyl iodide or hydroiodic acid in the reaction medium to generate the corresponding co-promoter iodide salt stabilizer. For additional detail regarding rhodium catalysis and iodide salt generation, see U.S. Pat. Nos. 5,001,259; 5,026,908; and 5,144,068, which are hereby incorporated by reference.

When an iridium catalyst is utilized, the iridium catalyst may comprise any iridium-containing compound which is soluble in the liquid reaction composition. The iridium catalyst may be added to the liquid reaction composition for the carbonylation reaction in any suitable form which dissolves in the liquid reaction composition or is convertible to a soluble form. Examples of suitable iridium-containing compounds which may be added to the liquid reaction composition include: IrCl₃, IrI₃, IrBr₃, [Ir(CO)₂I]₂, [Ir(CO)₂Cl]₂, [Ir(CO)₂Br]₂, [Ir(CO)₂I₂]⁻H⁺, [Ir(CO)₂Br₂]⁻H⁺, [Ir(CO)₂I₄]⁻H⁺, [Ir(CH₃)I₃(CO₂]⁻H⁺, Ir₄(CO)₁₂, IrCl₃.3H₂O, IrBr₃.3H₂O, Ir₄(CO)₁₂, iridium metal, Ir₂O₃, Ir(acac)(CO)₂, Ir(acac)₃, iridium acetate, [Ir₃O(OAc)₆(H₂O)₃][OAc], and hexachloroiridic acid [H₂IrCl₆]. Chloride-free complexes of iridium such as acetates, oxalates and acetoacetates are usually employed as starting materials. The iridium catalyst concentration in the liquid reaction composition may be in the range of 100 to 6000 ppm. The carbonylation of methanol utilizing iridium catalyst is well known and is generally described in U.S. Pat. Nos. 5,942,460; 5,932,764; 5,883,295; 5,877,348; 5,877,347; and 5,696,284, which are hereby incorporated by reference.

A halogen co-catalyst/promoter is generally used in combination with the Group VIII metal catalyst component. Methyl iodide is a preferred halogen promoter. Preferably, the concentration of halogen promoter in the reaction medium ranges from 1 wt. % to 50 wt. %, and preferably from 2 wt. % to 30 wt. %. The halogen-containing catalyst promoter of the catalyst system comprises a halogen compound, typically an organic halide. Thus, alkyl, aryl, and substituted alkyl or aryl halides can be used. Preferably, the halogen-containing catalyst promoter is present in the form of an alkyl halide. Even more preferably, the halogen-containing catalyst promoter is present in the form of an alkyl halide in which the alkyl radical corresponds to the alkyl radical of the feed alcohol, which is being carbonylated. Thus, in the carbonylation of methanol to acetic acid, the halide promoter will include methyl halide, and more preferably methyl iodide.

The halogen promoter may be combined with the salt stabilizer/co-promoter compound. Particularly preferred are iodide or acetate salts, e.g., lithium iodide or lithium acetate.

Other promoters and co-promoters may be used as part of the catalytic system of the present invention as described in U.S. Pat. No. 5,877,348, which is hereby incorporated by reference. Suitable promoters are selected from ruthenium, osmium, tungsten, rhenium, zinc, cadmium, indium, gallium, mercury, nickel, platinum, vanadium, titanium, copper, aluminum, tin, antimony, and are more preferably selected from ruthenium and osmium. Specific co-promoters are described in U.S. Pat. No. 6,627,770, which is incorporated herein by reference.

A promoter may be present in an effective amount up to the limit of its solubility in the liquid reaction composition and/or any liquid process streams recycled to the carbonylation reactor from the acetic acid recovery stage. When used, the promoter is suitably present in the liquid reaction composition at a molar ratio of promoter to metal catalyst of 0.5:1 to 15:1, preferably 2:1 to 10:1, more preferably 2:1 to 7.5:1. A suitable promoter concentration is 400 to 5000 ppm.

In one embodiment, the temperature of the carbonylation reaction in the reactor is preferably from 150° C. to 250° C., e.g., from 150° C. to 225° C., or from 150° C. to 200° C. The pressure of the carbonylation reaction is preferably from 1 to 20 MPa, preferably 1 to 10 MPa, most preferably 1.5 to 5 MPa. Acetic acid is typically manufactured in a liquid phase reaction at a temperature from about 150° C. to about 200° C. and a total pressure of from about 2 to about 5 MPa.

The liquid reaction medium employed may include any solvent compatible with the catalyst system and may include pure alcohols, or mixtures of the alcohol feedstock and/or the desired carboxylic acid and/or esters of these two compounds. A preferred solvent and liquid reaction medium for the low water carbonylation process contains the desired carboxylic acid product. Thus, in the carbonylation of methanol to acetic acid, a preferred solvent system contains acetic acid.

In one embodiment, reaction mixture comprises a reaction solvent or mixture of solvents. The solvent is preferably compatible with the catalyst system and may include pure alcohols, mixtures of an alcohol feedstock, and/or the desired carboxylic acid and/or esters of these two compounds. In one embodiment, the solvent and liquid reaction medium for the (low water) carbonylation process is preferably acetic acid.

Water may be formed in situ in the reaction medium, for example, by the esterification reaction between methanol reactant and acetic acid product. In some embodiments, water is introduced to reactor together with or separately from other components of the reaction medium. Water may be separated from the other components of reaction product withdrawn from reactor and may be recycled in controlled amounts to maintain the required concentration of water in the reaction medium. Preferably, the concentration of water maintained in the reaction medium ranges from 0.1 wt. % to 16 wt. %, e.g., from 1 wt. % to 14 wt. %, or from 1 wt. % to 3 wt. % of the total weight of the reaction product.

The desired reaction rates are obtained even at low water concentrations by maintaining in the reaction medium an ester of the desired carboxylic acid and an alcohol, desirably the alcohol used in the carbonylation, and an additional iodide ion that is over and above the iodide ion that is present as hydrogen iodide. An example of a preferred ester is methyl acetate. The additional iodide ion is desirably an iodide salt, with lithium iodide (LiI) being preferred. It has been found, as described in U.S. Pat. No. 5,001,259, that under low water concentrations, methyl acetate and lithium iodide act as rate promoters only when relatively high concentrations of each of these components are present and that the promotion is higher when both of these components are present simultaneously. The absolute concentration of iodide ion content is not a limitation on the usefulness of the present invention.

In low water carbonylation, the additional iodide, as supplement to the organic iodide promoter may be present in the catalyst solution in amounts ranging from 2 wt. % to 20 wt. %, e.g., from 2 wt. % to 15 wt. %, or from 3 wt. % to 10 wt. %; the methyl acetate may be present in amounts ranging from 0.5 wt % to 30 wt. %, e.g., from 1 wt. % to 25 wt. %, or from 2 wt. % to 20 wt. %; and the lithium iodide may be present in amounts ranging from 5 wt. % to 20 wt. %, e.g., from 5 wt. % to 15 wt. %, or from 5 wt. % to 10 wt. %. The catalyst may be present in the catalyst solution in amounts ranging from 200 wppm to 2000 wppm, e.g., from 200 wppm to 1500 wppm, or from 500 wppm to 1500 wppm.

The crude acetic acid stream may be separated to form a purified acetic acid stream. Exemplary purification schemes are discussed below.

### Condensation Reaction

As stated above, the carbonylation process may be integrated with a condensation process. The condensation process may react the alkanoic acid, e.g., acetic acid, from the carbonylation reaction with an alkylenating agent to produce acrylate products. The following reaction conditions and catalysts are exemplary.

The acetic acid, along with water, may be vaporized at the reaction temperature, following which the vaporized acetic acid can be fed along with alkylenating agent in an undiluted state or diluted with water or a relatively inert carrier gas, such as nitrogen, argon, helium, carbon dioxide and the like. For reactions run in the vapor phase, the temperature should be controlled in the system such that it does not fall below the dew point of the gas mixture in the reactor. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing followed by heating the mixed vapors up to the reactor inlet temperature. Preferably, the acetic acid and/or the alkylenating agent are vaporized using heat streams from the carbonylation reaction. By using these heat streams as a heat source, the cost for vaporizing the acetic acid and/or the alkylenating agent may be reduced.

The inventive process, in one embodiment, yields a crude acrylate product stream comprising the acrylic acid and/or other acrylate products. The crude acrylate product stream of the present invention, unlike most conventional acrylic acid-containing crude products, further comprises a significant portion of at least one alkylenating agent. Preferably, the at least one alkylenating agent is formaldehyde. For example, the crude product stream may comprise at least 1 wt. % alkylenating agent(s), e.g., at least 3 wt. %, at least 5 wt. %, at least 7 wt. %, at least 10 wt. %, or at least 25 wt. %. In terms of ranges, the crude product stream may comprise from 1 wt. % to 50 wt. % alkylenating agent(s), e.g., from 1 wt. % to 45 wt. %, from 1 wt. % to 25 wt. %, from 1 wt. % to 10 wt. %, or from 5 wt. % to 10 wt. %. In terms of upper limits, the crude product stream may comprise less than 50 wt. % alkylenating agent(s), e.g., less than 45 wt. %, less than 25 wt. %, or less than 10 wt. %.

In one embodiment, the crude acrylate product stream of the present invention further comprises water, which may not just come as the co-product of acrylic acid in the aldol condensation reaction. In one embodiment, a portion of the water present in the crude acrylate product may come from crude acetic acid. In some embodiments, a portion of the water in the crude acrylate product may come from the alkylenating agent stream. As mentioned before, the condensation of acetic acid and formaldehyde also generates additional water. For example, the crude acrylate product stream may comprise more than 5 wt. % water, e.g., more than 10 wt. %, or more than 18 wt. %. In terms of ranges, the crude acrylate product stream may comprise from 5 wt. % to 80 wt. % water, e.g., from 10 wt. % to 70 wt. %, or from 18 wt. % to 60 wt. %. In terms of lower limits, the crude acrylate product stream may comprise at least 1 wt. % water, e.g., at least 5 wt. %, at least 10 wt. %, or at least 15 wt. %.

In one embodiment, the crude product stream of the present invention comprises very little, if any, of the impurities found in most conventional acrylic acid crude product streams. For example, the crude product stream of the present invention may comprise less than 1000 wppm of such impurities (either as individual components or collectively), e.g., less than 500 wppm, less than 100 wppm, less than 50 wppm, or less than 10 wppm. Exemplary impurities include acetylene, ketene, beta-propiolactone, higher alcohols, e.g., C₂₊, C₃₊, or C₄₊, and combinations thereof. Importantly, the crude product stream of the present invention comprises very little, if any, furfural and/or acrolein. In one embodiment, the crude product stream comprises substantially no furfural and/or acrolein, e.g., no furfural and/or acrolein. In one embodiment, the crude product stream comprises less than less than 500 wppm acrolein, e.g., less than 100 wppm, less than 50 wppm, or less than 10 wppm. In one embodiment, the crude product stream comprises less than less than 500 wppm furfural, e.g., less than 100 wppm, less than 50 wppm, or less than 10 wppm. Furfural and acrolein are known to act as detrimental chain terminators in acrylic acid polymerization reactions. Also, furfural and/or acrolein are known to have adverse effects on the color of purified product and/or to subsequent polymerized products.

In addition to the acrylic acid and the alkylenating agent, the crude acrylate product stream may further comprise acetic acid, water, propionic acid, and light ends such as oxygen, nitrogen, carbon monoxide, carbon dioxide, methanol, methyl acetate, methyl acrylate, acetaldehyde, hydrogen, and acetone. Exemplary compositional data for the crude product stream are shown in Table 1. Components other than those listed in Table 1 may also be present in the crude product stream.

TABLE 1
CRUDE ACRYLATE PRODUCT STREAM COMPOSITIONS

| | | | |
|---|---|---|---|
| Acrylic Acid | 1 to 75 | 1 to 50 | 5 to 50 |
| Alkylenating Agent(s) | 0.5 to 50 | 1 to 45 | 1 to 25 |
| Acetic Acid | 1 to 90 | 1 to 70 | 5 to 50 |
| Water | 1 to 60 | 5 to 50 | 5 to 40 |
| Propionic Acid | 0.01 to 10 | 0.1 to 10 | 0.1 to 5 |
| Oxygen | 0.01 to 20 | 0.1 to 10 | 0.1 to 5 |
| Nitrogen | 0.1 to 80 | 0.1 to 60 | 0.5 to 40 |
| Carbon Monoxide | 0.01 to 35 | 0.1 to 25 | 0.1 to 15 |
| Carbon Dioxide | 0.01 to 30 | 0.1 to 20 | 0.1 to 10 |
| Other Light Ends | 0.01 to 30 | 0.1 to 20 | 0.1 to 10 |

The unique crude acrylate product stream of the present invention may be separated in a separation zone to form a final product, e.g., a final acrylic acid product.

In one embodiment, the inventive process operates at a high process efficiency. For example, the process efficiency may be at least 10%, e.g., at least 20% or at least 35%. In one embodiment, the process efficiency is calculated based on the flows of reactants into the reaction zone. The process efficiency may be calculated by the following formula.

Process Efficiency=2N_{HAcA}/[N_{HOAc}+N_{HCHO}+N_{H2O}]

where:

N_{HAcA} is the molar production rate of acrylate products; and

N_{HOAc}, N_{HCHO}, and N_{H2O} are the molar feed rates of acetic acid, formaldehyde, and water.

In terms of the production of acrylate products, any suitable reaction and/or separation scheme may be employed to faun the crude product stream as long as the reaction provides the crude product stream components that are discussed above. For example, in some embodiments, the acrylate product stream is formed by contacting an alkanoic acid, e.g., acetic acid, or an ester thereof with an alkylenating agent, e.g., a methylenating agent, for example formaldehyde, under conditions effective to form the crude acrylate product stream. Preferably, the contacting is performed over a suitable catalyst. The crude product stream may be the reaction product of the alkanoic acid-alkylenating agent reaction. In a preferred embodiment, the crude product stream is the reaction product of the aldol condensation reaction of acetic acid and formaldehyde, which is conducted over a catalyst comprising vanadium and titanium. In one embodiment, the crude product stream is the product of a reaction wherein methanol and acetic acid are combined to generate at least a portion of formaldehyde in situ. Unreacted methanol from the carbonylation reaction may be carried over in the crude acetic acid/water stream and at least a portion of formaldehyde may be generated therefrom. The aldol condensation then follows. In one embodiment, a methanol-formaldehyde solution is reacted with acetic acid to form the crude product stream.

In some embodiments, the condensation reaction may achieve favorable conversion of acetic acid and favorable selectivity and productivity to acrylates. For purposes of the present invention, the term “conversion” refers to the amount of acetic acid in the feed that is converted to a compound other than acetic acid. Conversion is expressed as a percentage based on acetic acid in the feed. The conversion of acetic acid may be at least 10%, e.g., at least 20%, at least 40%, or at least 50%.

Selectivity, as it refers to the formation of acrylate product, is expressed as the ratio of the amount of carbon in the desired product(s) and the amount of carbon in the total products. This ratio may be multiplied by 100 to arrive at the selectivity. Preferably, the catalyst selectivity to acrylate products, e.g., acrylic acid and methyl acrylate, is at least 40 mol %, e.g., at least 50 mol %, at least 60 mol %, or at least 70 mol %. In some embodiments, the selectivity to acrylic acid is at least 30 mol %, e.g., at least 40 mol %, or at least 50 mol %; and/or the selectivity to methyl acrylate is at least 10 mol %, e.g., at least 15 mol %, or at least 20 mol %.

The terms “productivity” or “space time yield” as used herein, refers to the grams of a specified product, e.g., acrylate products, formed per hour during the condensation based on the liters of catalyst used. A productivity of at least 20 grams of acrylate product per liter catalyst per hour, e.g., at least 40 grams of acrylates per liter catalyst per hour or at least 100 grams of acrylates per liter catalyst per hour, is preferred. In terms of ranges, the productivity preferably is from 20 to 500 grams of acrylates per liter catalyst per hour, e.g., from 20 to 200 grams of acrylates per liter catalyst per hour or from 40 to 140 grams of acrylates per liter catalyst per hour.

In one embodiment, the inventive process yields at least 1,800 kg/hr of finished acrylic acid, e.g., at least 3,500 kg/hr, at least 18,000 kg/hr, or at least 37,000 kg/hr.

Preferred embodiments of the inventive process demonstrate a low selectivity to undesirable products, such as carbon monoxide and carbon dioxide. The selectivity to these undesirable products preferably is less than 29%, e.g., less than 25% or less than 15%. More preferably, these undesirable products are not detectable. Formation of alkanes, e.g., ethane, may be low, and ideally less than 2%, less than 1%, or less than 0.5% of the acetic acid passed over the catalyst is converted to alkanes, which have little value other than as fuel.

The crude acetic acid stream from the carbonylation reaction and alkylenating agent may be fed independently or after prior mixing to a reactor containing the catalyst. The reactor may be any suitable reactor or combination of reactors. Preferably, the reactor comprises a fixed bed reactor or a series of fixed bed reactors. In one embodiment, the reactor is a packed bed reactor or a series of packed bed reactors. In one embodiment, the reactor is a fixed bed reactor. Of course, other reactors such as a continuous stirred tank reactor or a fluidized bed reactor may be employed.

In some embodiments, the alkanoic acid, e.g., crude acetic acid stream, and the alkylenating agent, e.g., formaldehyde, are fed to the reactor at a molar ratio of at least 0.10:1, e.g., at least 0.75:1, at least 1:1, or at least 1.5:1. In terms of ranges the molar ratio of alkanoic acid to alkylenating agent may range from 0.10:1 to 10:1 or from 0.75:1 to 5:1. In some embodiments, the reaction of the alkanoic acid and the alkylenating agent is conducted with a stoichiometric excess of alkanoic acid. In these instances, catalyst performances, like acrylate selectivity, may be improved. As an example the acrylate selectivity may be at least 10% higher than a selectivity achieved when the reaction is conducted with an excess of alkylenating agent, e.g., at least 20% higher or at least 30% higher. In other embodiments, the reaction of the alkanoic acid and the alkylenating agent is conducted with a stoichiometric excess of alkylenating agent.

The condensation reaction may be conducted at a temperature of at least 250° C., e.g., at least 300° C., or at least 350° C. In terms of ranges, the reaction temperature may range from 200° C. to 500° C., e.g., from 250° C. to 400° C., or from 250° C. to 350° C. Residence time in the reactor may range from 0.1 second to 200 seconds, e.g., from 1 second to 100 seconds. Reaction pressure is not particularly limited, and the reaction is typically performed near atmospheric pressure. In one embodiment, the reaction may be conducted at a pressure ranging from 0 kPa to 4100 kPa, e.g., from 3 kPa to 345 kPa, or from 6 kPa to 103 kPa. The acetic acid conversion, in some embodiments, may vary depending upon the reaction temperature and other operating parameters

In one embodiment, the reaction is conducted at a gas hourly space velocity (“GHSV”) greater than 600 hr⁻¹, e.g., greater than 1000 hr⁻¹ or greater than 2000 hr⁻¹. In one embodiment, the GHSV ranges from 600 hr⁻¹ to 10000 hr⁻¹, e.g., from 1000 hr⁻¹ to 8000 hr⁻¹ or from 1500 hr⁻¹ to 7500 hr⁻¹. As one particular example, when GHSV is at least 2000 hr⁻¹, the acrylate product space time yield (STY) may be at least 150 g/hr/liter.

In one embodiment, water may be present in the reactor in amounts up to 60 wt. % of the reaction mixture, e.g., up to 50 wt. % or up to 40 wt. %. The additional water from the acetic acid feed streams does not negatively impact the production of acrylate product. In addition, the costs of removing water from the crude acrylate product may be mitigated by the integration of heated streams from the carbonylation reaction as an energy source. Thus, the overall operating cost of the production of the acrylate product may be reduced.

When the desired product is an unsaturated ester made by reacting an ester of an alkanoic acid ester with formaldehyde, the alcohol corresponding to the ester may also be fed to the reactor either with or separately to the other components. For example, when methyl acrylate is desired, methanol may be fed to the reactor. The alcohol, amongst other effects, reduces the quantity of acids leaving the reactor. It is not necessary that the alcohol is added at the beginning of the reactor and it may for instance be added in the middle or near the back, in order to effect the conversion of acids such as propionic acid, methacrylic acid to their respective esters without depressing catalyst activity. In one embodiment, the alcohol may be added downstream of the reactor.

The condensation of the alkanoic acid and alkylenating agent is preferably conducted in the presence of a condensation catalyst. The catalyst may be any suitable catalyst composition. As one example, condensation catalyst consisting of mixed oxides of vanadium and phosphorus have been investigated and described in M. Ai, J. Catal., 107, 201 (1987); M. Ai, J. Catal., 124, 293 (1990); M. Ai, Appl. Catal., 36, 221 (1988); and M. Ai, Shokubai, 29, 522 (1987). Other examples include binary vanadium-titanium phosphates, vanadium-silica-phosphates, and alkali metal-promoted silicas, e.g., cesium- or potassium-promoted silicas.

In a preferred embodiment, the inventive process employs a catalyst composition comprising vanadium, titanium, and optionally at least one oxide additive. The oxide additive(s), if present, are preferably present in the active phase of the catalyst. In one embodiment, the oxide additive(s) are selected from the group consisting of silica, alumina, zirconia, and mixtures thereof or any other metal oxide other than metal oxides of titanium or vanadium. Preferably, the molar ratio of oxide additive to titanium in the active phase of the catalyst composition is greater than 0.05:1, e.g., greater than 0.1:1, greater than 0.5:1, or greater than 1:1. In terms of ranges, the molar ratio of oxide additive to titanium in the inventive catalyst may range from 0.05:1 to 20:1, e.g., from 0.1:1 to 10:1, or from 1:1 to 10:1. In these embodiments, the catalysts comprise titanium, vanadium, and one or more oxide additives and have relatively high molar ratios of oxide additive to titanium.

In other embodiments, the catalyst may further comprise other compounds or elements (metals and/or non-metals). For example, the catalyst may further comprise phosphorus and/or oxygen. In these cases, the catalyst may comprise from 15 wt. % to 45 wt. % phosphorus, e.g., from 20 wt. % to 35 wt. % or from 23 wt. % to 27 wt. %; and/or from 30 wt. % to 75 wt. % oxygen, e.g., from 35 wt. % to 65 wt. % or from 48 wt. % to 51 wt. %.

In some embodiments, the catalyst further comprises additional metals and/or oxide additives. These additional metals and/or oxide additives may function as promoters. If present, the additional metals and/or oxide additives may be selected from the group consisting of copper, molybdenum, tungsten, nickel, niobium, and combinations thereof. Other exemplary promoters that may be included in the catalyst of the invention include lithium, sodium, magnesium, aluminum, chromium, manganese, iron, cobalt, calcium, yttrium, ruthenium, silver, tin, barium, lanthanum, the rare earth metals, hafnium, tantalum, rhenium, thorium, bismuth, antimony, germanium, zirconium, uranium, cesium, zinc, and silicon and mixtures thereof. Other modifiers include boron, gallium, arsenic, sulfur, halides, Lewis acids such as BF₃, ZnBr₂, and SnCl₄. Exemplary processes for incorporating promoters into catalyst are described in U.S. Pat. No. 5,364,824, the entirety of which is incorporated herein by reference.

In one embodiment, the catalyst comprises bismuth. In one embodiment, the catalyst comprises tungsten. In one embodiment, the catalyst comprises bismuth and tungsten. Preferably, the bismuth and/or the tungsten are employed with vanadium and/or titanium

If the catalyst comprises additional metal(s) and/or metal oxides(s), the catalyst optionally may comprise additional metals and/or metal oxides in an amount from 0.001 wt. % to 30 wt. %, e.g., from 0.01 wt. % to 5 wt. % or from 0.1 wt. % to 5 wt. %. If present, the promoters may enable the catalyst to have a weight/weight space time yield of at least 25 grams of acrylic acid/gram catalyst-h, e.g., least 50 grams of acrylic acid/gram catalyst-h, or at least 100 grams of acrylic acid/gram catalyst-h.

In some embodiments, the catalyst is unsupported. In these cases, the catalyst may comprise a homogeneous mixture or a heterogeneous mixture as described above. In one embodiment, the homogeneous mixture is the product of an intimate mixture of vanadium and titanium oxides, hydroxides, and phosphates resulting from preparative methods such as controlled hydrolysis of metal alkoxides or metal complexes. In other embodiments, the heterogeneous mixture is the product of a physical mixture of the vanadium and titanium phosphates. These mixtures may include formulations prepared from phosphorylating a physical mixture of preformed hydrous metal oxides. In other cases, the mixture(s) may include a mixture of preformed vanadium pyrophosphate and titanium pyrophosphate powders.

In another embodiment, the catalyst is a supported catalyst comprising a catalyst support in addition to the vanadium, titanium, oxide additive, and optionally phosphorous and oxygen, in the amounts indicated above (wherein the molar ranges indicated are without regard to the moles of catalyst support, including any vanadium, titanium, oxide additive, phosphorous or oxygen contained in the catalyst support). The total weight of the support (or modified support), based on the total weight of the catalyst, preferably is from 75 wt. % to 99.9 wt. %, e.g., from 78 wt. % to 97 wt. % or from 80 wt. % to 95 wt. %. The support may vary widely. In one embodiment, the support material is selected from the group consisting of silica, alumina, zirconia, titania, aluminosilicates, zeolitic materials, mixed metal oxides (including but not limited to binary oxides such as SiO₂—Al₂O₃, SiO₂—TiO₂, SiO₂—ZnO, SiO₂—MgO, SiO₂—ZrO₂, Al₂O₃—MgO, Al₂O₃—TiO₂, Al₂O₃—ZnO, TiO₂—MgO, TiO₂—ZrO₂, TiO₂—ZnO, TiO₂—SnO₂) and mixtures thereof, with silica being one preferred support. In embodiments where the catalyst comprises a titania support, the titania support may comprise a major or minor amount of rutile and/or anatase titanium dioxide. Other suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, silicon carbide, sheet silicates or clay minerals such as montmorillonite, beidellite, saponite, pillared clays, other microporous and mesoporous materials, and mixtures thereof. Other supports may include, but are not limited to, iron oxide, magnesia, steatite, magnesium oxide, carbon, graphite, high surface area graphitized carbon, activated carbons, and mixtures thereof. These listings of supports are merely exemplary and are not meant to limit the scope of the present invention.

In some embodiments, a zeolitic support is employed. For example, the zeolitic support may be selected from the group consisting of montmorillonite, NH4 ferrierite, H-mordenite-PVOx, vermiculite-1, H-ZSM5, NaY, H-SDUSY, Y zeolite with high SAR, activated bentonite, H-USY, MONT-2, HY, mordenite SAR 20, SAPO-34, Aluminosilicate (X), VUSY, Aluminosilicate (CaX), Re-Y, and mixtures thereof. H-SDUSY, VUSY, and H-USY are modified Y zeolites belonging to the faujasite family. In one embodiment, the support is a zeolite that does not contain any metal oxide modifier(s). In some embodiments, the catalyst composition comprises a zeolitic support and the active phase comprises a metal selected from the group consisting of vanadium, aluminum, nickel, molybdenum, cobalt, iron, tungsten, zinc, copper, titanium cesium bismuth, sodium, calcium, chromium, cadmium, zirconium, and mixtures thereof. In some of these embodiments, the active phase may also comprise hydrogen, oxygen, and/or phosphorus.

In other embodiments, in addition to the active phase and a support, the inventive catalyst may further comprise a support modifier. A modified support, in one embodiment, relates to a support that includes a support material and a support modifier, which, for example, may adjust the chemical or physical properties of the support material such as the acidity or basicity of the support material. In embodiments that use a modified support, the support modifier is present in an amount from 0.1 wt. % to 50 wt. %, e.g., from 0.2 wt. % to 25 wt. %, from 0.5 wt. % to 15 wt. %, or from 1 wt. % to 8 wt. %, based on the total weight of the catalyst composition.

In one embodiment, the support modifier is an acidic support modifier. In some embodiments, the catalyst support is modified with an acidic support modifier. The support modifier similarly may be an acidic modifier that has a low volatility or little volatility. The acidic modifiers may be selected from the group consisting of oxides of Group IVB metals, oxides of Group VB metals, oxides of Group VIB metals, iron oxides, aluminum oxides, and mixtures thereof. In one embodiment, the acidic modifier may be selected from the group consisting of WO₃, MoO₃, Fe₂O₃, Cr₂O₃, V₂O₅, MnO₂, CuO, Co₂O₃, Bi₂O₃, TiO₂, ZrO₂, Nb₂O₅, Ta₂O₅, Al₂O₃, B₂O₃, P₂O₅, and Sb₂O₃.

In another embodiment, the support modifier is a basic support modifier. The presence of chemical species such as alkali and alkaline earth metals, are normally considered basic and may conventionally be considered detrimental to catalyst performance. The presence of these species, however, surprisingly and unexpectedly, may be beneficial to the catalyst performance. In some embodiments, these species may act as catalyst promoters or a necessary part of the acidic catalyst structure such in layered or sheet silicates such as montmorillonite. Without being bound by theory, it is postulated that these cations create a strong dipole with species that create acidity.

Additional modifiers that may be included in the catalyst include, for example, boron, aluminum, magnesium, zirconium, and hafnium.

As will be appreciated by those of ordinary skill in the art, the support materials, if included in the catalyst of the present invention, preferably are selected such that the catalyst system is suitably active, selective and robust under the process conditions employed for the formation of the desired product, e.g., acrylic acid or alkyl acrylate. Also, the active metals and/or pyrophosphates that are included in the catalyst of the invention may be dispersed throughout the support, coated on the outer surface of the support (egg shell) or decorated on the surface of the support. In some embodiments, in the case of macro- and meso-porous materials, the active sites may be anchored or applied to the surfaces of the pores that are distributed throughout the particle and hence are surface sites available to the reactants but are distributed throughout the support particle.

The inventive catalyst may further comprise other additives, examples of which may include: molding assistants for enhancing moldability; reinforcements for enhancing the strength of the catalyst; pore-forming or pore modification agents for formation of appropriate pores in the catalyst, and binders. Examples of these other additives include stearic acid, graphite, starch, cellulose, silica, alumina, glass fibers, silicon carbide, and silicon nitride. Preferably, these additives do not have detrimental effects on the catalytic performances, e.g., conversion and/or activity. These various additives may be added in such an amount that the physical strength of the catalyst does not readily deteriorate to such an extent that it becomes impossible to use the catalyst practically as an industrial catalyst.

In one embodiment, one or more guard beds (not shown) may be used upstream of the reactor to protect the catalyst from poisons or undesirable impurities contained in the feed or return/recycle streams. Such guard beds may be employed in the vapor or liquid streams. Suitable guard bed materials may include, for example, carbon, silica, alumina, ceramic, or resins. In one aspect, the guard bed media is functionalized, e.g., silver functionalized, to trap particular species such as sulfur or halogens.

As noted above, the presence of alkylenating agent in the crude product stream adds unpredictability and problems to separation schemes. Without being bound by theory, it is believed that formaldehyde reacts in many side reactions with water to form by-products. The following side reactions are exemplary.

CH₂O+H₂O→HOCH₂OH

HO(CH₂O)ᵢ₋₁H+HOCH₂OH→HO(CH₂O)ᵢH+H₂O for i>1

Without being bound by theory, it is believed that, in some embodiments, as a result of these reactions, the alkylenating agent, e.g., formaldehyde, acts as a “light” component at higher temperatures and as a “heavy” component at lower temperatures. The reaction(s) are exothermic. Accordingly, the equilibrium constant increases as temperature decreases and decreases as temperature increases. At lower temperatures, the larger equilibrium constant favors methylene glycol and oligomer production and formaldehyde becomes limited, and, as such, behaves as a heavy component. At higher temperatures, the smaller equilibrium constant favors formaldehyde production and methylene glycol becomes limited. As such, formaldehyde behaves as a light component. In view of these difficulties, as well as others, the separation of streams that comprise water and formaldehyde cannot be expected to behave as a typical two-component system. These features contribute to the unpredictability and difficulty of the separation of the unique crude product stream of the present invention.

The present invention, surprisingly and unexpectedly, achieves effective separation of alkylenating agent(s) from the inventive crude product stream to yield a purified product comprising acrylate product and very low amounts of other impurities. Exemplary separation schemes are discussed herein.

In one embodiment, the alkylenating split is performed such that a lower amount of acetic acid is present in the resulting alkylenating stream. Preferably, the alkylenating agent stream comprises little or no acetic acid. As an example, the alkylenating agent stream, in some embodiments, comprises less than 50 wt. % acetic acid, e.g., less than 45 wt. %, less than 25 wt. %, less than 10 wt. %, less than 5 wt. %, less than 3 wt. %, or less than 1 wt. %. Surprisingly and unexpectedly, the present invention provides for the lower amounts of acetic acid in the alkylenating agent stream, which, beneficially reduces or eliminates the need for further treatment of the alkylenating agent stream to remove acetic acid. In some embodiments, the alkylenating agent stream may be treated to remove water therefrom, e.g., to purge water.

In some embodiments, the alkylenating agent split is performed in at least one column, e.g., at least two columns or at least three columns. Preferably, the alkylenating agent is performed in a two column system. In other embodiments, the alkylenating agent split is performed via contact with an extraction agent. In other embodiments, the alkylenating agent split is performed via precipitation methods, e.g., crystallization, and/or azeotropic distillation. Of course, other suitable separation methods may be employed either alone or in combination with the methods mentioned herein.

As mentioned above, the crude acrylate product stream of the present invention comprises little, if any, furfural and/or acrolein. As such the derivative stream(s) of the crude product streams will comprise little, if any, furfural and/or acrolein. In one embodiment, the derivative stream(s), e.g., the streams of the separation zone, comprises less than less than 500 wppm acrolein, e.g., less than 100 wppm, less than 50 wppm, or less than 10 wppm. In one embodiment, the derivative stream(s) comprises less than less than 500 wppm furfural, e.g., less than 100 wppm, less than 50 wppm, or less than 10 wppm.

### Carbonylation and Condensation Integration

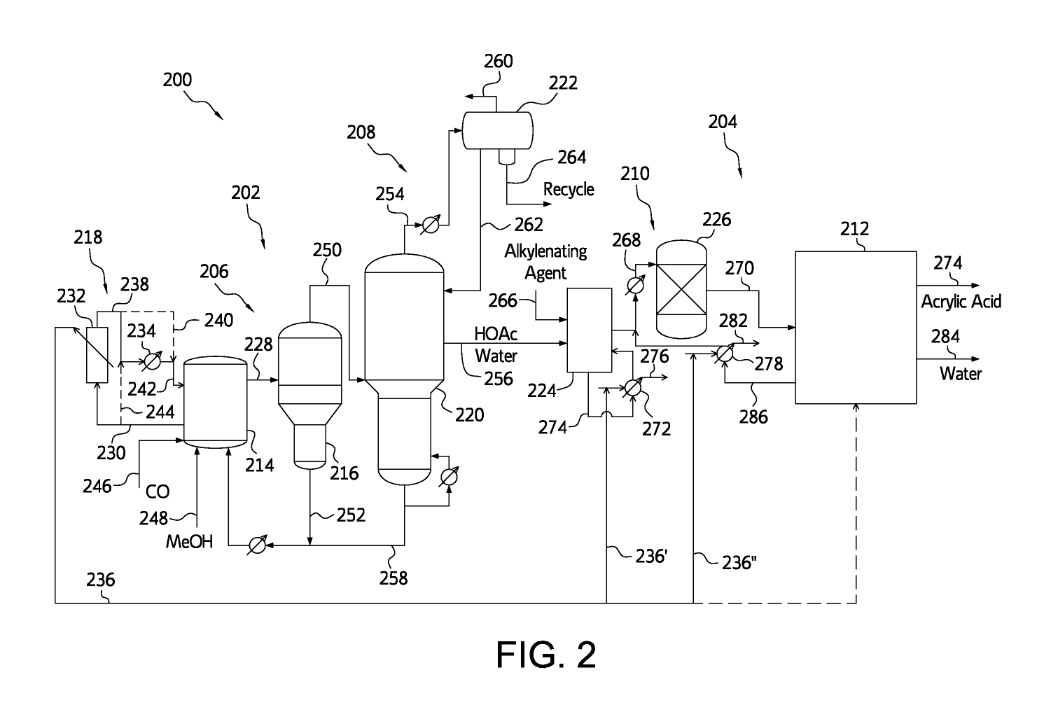

FIG. 2 shows exemplary integrated carbonylation and condensation process 200, which comprises carbonylation system 202 and condensation system 204. Carbonylation system 202 comprises 1) carbonylation reaction zone 206, which comprises carbonylation reactor 214, flasher 216, and heat transfer system 218, and 2) carbonylation separation zone 208, which comprises at least one distillation column, e.g., a light ends column or a drying column, 220, and phase separator, e.g., decanter, 222. Condensation system 204 comprises 1) condensation reaction zone 210, which comprises vaporizer 224 and condensation reactor 226 and 2) condensation separation zone 212, which comprises at least one distillation column (not shown).

Acrylate product separation zone 212 may also comprise an optional light ends removal unit (not shown). For example, the light ends removal unit may comprise a condenser and/or a flasher. The light ends removal unit may be configured either upstream or downstream of the alkylenating agent split unit. Depending on the configuration, the light ends removal unit removes light ends as well as non-condensable gases from the crude product stream, the alkylenating stream, and/or the intermediate acrylate product stream. In one embodiment, when the light ends are removed, the remaining liquid phase comprises the acrylic acid, acetic acid, alkylenating agent, and/or water.

In carbonylation system 202, methanol feed stream 248 comprises methanol and/or reactive derivatives thereof and carbon monoxide 246 are fed to a lower portion of carbonylation reactor 214. In one embodiment, a vaporizer may be employed. Reactants in lines 246 and 248 may be combined and jointly fed to the vaporizer prior to being fed to carbonylation reactor 214.

Suitable reactive derivatives of methanol include methyl acetate, dimethyl ether, methyl formate, and mixtures thereof. At least some of the methanol and/or reactive derivative thereof will be converted to, and hence present as, methyl acetate in the liquid reaction composition by reaction with acetic acid product or solvent. The concentration in the liquid reaction composition of methyl acetate is suitably in the range of from 0.5 wt. % to 70 wt. %, e.g., from 0.5 wt. % to 50 wt. %, from 1 wt. % to 35 wt. %, or from 1 wt. % to 20 wt. %. In some embodiments, a small amount of water may be added to the carbonylation reactor to enhance the activity and stability of the reaction system. For example, less than 5 wt. % of additional water may be added, e.g., less than 3 wt. %, or less than 2 wt. %.

Reactor 214 is preferably either a stirred vessel, e.g., CSTR, or bubble-column type vessel, with agitator or without an agitator, within which the reaction medium is maintained, preferably automatically, at a predetermined level. This predetermined level may remain substantially constant during normal operation. Methanol, carbon monoxide, and sufficient water may be continuously introduced into reactor 214 as needed to maintain at least a finite concentration of water in the reaction medium. In one embodiment, carbon monoxide, e.g., in the gaseous state, is continuously introduced into reactor 214, desirably below agitator, which is used to enhance the gas dispersion and mass transfer of the contents. The temperature of reactor 214 may be controlled, as indicated above. Carbon monoxide feed 246 is introduced at a rate sufficient to maintain the desired total reactor pressure.

The gaseous carbon monoxide feed is preferably thoroughly dispersed through the reaction medium by agitator. A gaseous purge is desirably vented via an off-gas line (not shown) from reactor 214 to prevent buildup of gaseous by-products, such as methane, carbon dioxide, and hydrogen, and to maintain a carbon monoxide partial pressure at a given total reactor pressure.

Heat generated from carbonylation reactor 214 maybe conveyed to heat transfer system 218. As shown in FIG. 2, a reaction solution stream 230 may be taken directly from reactor 214 or optionally may be withdrawn from the carbonylation product stream 228 via a pump around loop (not shown). In operation, reactor solution stream 230 may be withdrawn at a temperature that is substantially similar to the reaction temperature and may be at a temperature from 150° C. to 250° C. Heat transfer system 218 may comprise one or more steam generator 232 and/or heat exchanger 234. For purposes of clarity one steam generator 232 and heat exchanger 234 are shown in FIG. 2. Additional steam generators and/or heat exchanges may be used in embodiments of the present invention. Heat transfer system 218 may also comprise pumps, variable speed electric motors and/or steam turbines, valves and controls for regulating the flow of the reaction solution stream 230 through heat transfer system 218.

In one embodiment, reactor solution stream 230 is preferably directed to steam generator 232 to produce steam condensate stream 236 and exiting process stream 238. Exiting process stream 238 may be returned directly to reactor via optional line 240 and return line 242. Reaction solution stream 230 comprises the components of the reaction medium and preferably is retained with the system and not discarded. After passing through steam generators, exiting process stream 238 may have a temperature below the carbonylation reaction temperature, e.g., below about 250° C., or from 150° C. to 200° C. In preferred embodiments, each pump around loop produces at least 1 tns/hr of steam, e.g., at least 3 tns/hr, 5 tns/hr or 10 tns/hr for acetic acid production at the rate of 25 tns/hr. In terms of ranges each around loop may produce from 3 to 10 tns/hr, e.g., from 3 to 5 tns/hr or 5 to 10 tns/hr at the rate of 25 tns/hr acetic acid production. In addition, in preferred embodiments, the steam produced may have variable qualities (pressure). The pressure may be at least 4 bars, e.g., at least 10 bars, or at least 20 bars. The quantity of steam produced by the steam generators from the heat transfer system 218 may vary based on the flow rate, control temperature in the carbonylation system reactor, condensate temperature, and the pressure quality of the steam being generated. Certain embodiments of the present invention enable the generation of high quantity, variable quality (i.e., pressure) steam to supply up to or even more than 100%, e.g., up to 80% or up to 50%, of steady state steam demand for the purification sections of the carbonylation system process.

Steam condensate stream 236 is conveyed to condensation system 204, e.g., to vaporizer 224, reactor 226 or separation zone 212. In addition, steam condensate stream 236 may be used to drive other systems in the carbonylation process such as turbine driven pumps and/or compressor, to flare, to heat storage tanks and/or buildings, to absorption refrigeration systems, etc. In some embodiments, steam condensate stream is directed to an external energy consuming process.

Suitable steam generators may include a shell and tube exchanger, double pipe exchanger, spiral plate exchanger, plate heat exchanger, helical coil, spiral coil or bayonet tube in tank heat exchanger, or any other suitable heat exchanger known in the art. The process side of the steam generator can be comprised of any suitable construction material known in the art, for example a nickel-molybdenum alloy such as HASTELLOY™ B-3 alloy (Haynes International) or a zirconium alloy such as Zirc™ 702 alloy (United Titanium Inc.). The steam (water) side of the steam generator can be comprised of any suitable construction metal, including carbon steel and lower grade stainless and alloy steels.

In one embodiment, reactor solution stream 230 may be directed to heat exchanger 234 to provide temperature regulation of reactor 214, via optional line 244. The outflow of heat exchanger 234 may be returned to reactor via return line 242. Any suitable indirect-contact heat exchangers, including two medium transfer type heat exchangers or three medium transfer type heat exchangers, that are capable of transferring heat by conduction may be used with embodiments of the present invention. Heat exchangers may include a shell and tube exchanger, spiral plate heat exchanger, helical coil exchanger, or any other suitable heat exchanger known in the art. Sensible cooling heat exchangers are preferred. These heat exchangers preferably provide bulk and/or trim cooling to remove the excess heat of the reaction from the carbonylation reaction of the system. In addition, in some embodiments, heat exchangers may also produce steam. In still other embodiments, heat exchangers are used to provide heat to reactor 214 during start up and steam generator 232 may be bypassed by optional line 244. After passing through one of the heat exchangers in cooling mode, the outflow may have a temperature below the carbonylation reaction temperature, e.g., below about 200° C., or from 130° C. to 175° C.

In addition, in some embodiments, steam generator 232 may also provide temperature regulation of the carbonylation reactor with or without producing steam. Steam generator and heat exchanger may be used in combination to provide temperature regulation. For example, when the reactor is cooled about a third of the cooling may be provided by the steam generator and the remaining cooling provided by the heat exchanger.

In a preferred embodiment, reactor solution stream 230 is fed to steam generator 232 and a portion of exiting process stream 238 is directed to heat exchanger 234 to provide cooling of the reactor 214. The outflow of heat exchanger 234 may be returned to reactor 214 via line 242. Preferably reaction solution stream 230 is withdrawn and return line 242 is fed to reactor below the liquid level in reactor 214. In some embodiments, reaction solution stream 230 is withdrawn below the level at which carbonylation product 228 is withdrawn from the reactor 214. In one embodiment, reaction solution stream 230 and return line 242 may be connected to reactor 214 at similar elevations but at differing orientations.

Reactor 214 contains a catalyst that is used in the reaction to form crude product stream, which is withdrawn, preferably continuously, from reactor 214 via line 228. The crude acetic acid product is drawn off from the reactor 214 at a rate sufficient to maintain a constant level therein and is provided to flasher 216 via stream 228.

In flasher 216, the crude acetic acid product is separated in a flash separation step to obtain a volatile (“vapor”) overhead stream 250 comprising acetic acid and a less volatile stream 252 comprising a catalyst-containing solution. The catalyst-containing solution comprises acetic acid containing the rhodium and the iodide salt along with lesser quantities of methyl acetate, methyl iodide, and water. The less volatile stream 252 preferably is recycled to reactor 214. Vapor overhead stream 250 also comprises methyl iodide, methyl acetate, water, unreacted CO, unreacted methanol, and permanganate reducing compounds (“PRCs”).

Overhead stream 250 from flasher 216 is directed to carbonylation separation zone 208. Carbonylation separation zone 208 comprises light ends column 220 and decanter 222. Carbonylation separation zone 208 may also comprise additional units, e.g., a drying column (if necessary), one or more columns for removing PRCs, heavy ends columns, extractors, etc.

In light ends column 220, stream 250 is separated to form low-boiling overhead vapor stream 254, sidestream 256, which comprises a purified acetic acid stream, and a high boiling residue stream 258. Purified acetic acid that is removed via sidestream 256 preferably is conveyed, e.g., directly, without removing substantially any water therefrom, to condensation system 204. Thus, the inventive condensation process provides for production efficiencies by using an acetic acid stream having a higher water content than glacial acetic acid, which beneficially reduces or eliminates the need for water removal downstream from light ends column 220 in carbonylation system 202.

In one embodiment, light ends column 220 may comprise trays having different concentrations of water. In these cases, the composition of a withdrawn sidedraw may vary throughout the column depending on the try location at which the sidedraw is withdrawn. As such, the withdrawal tray may be selected based on the amount of water that is desired, e.g., more than 0.5 wt. %. In another embodiment, the configuration of the column may be varied to achieve a desired amount or concentration of water in a sidedraw. Thus, an acetic acid feed stream may be produced, e.g., withdrawn from a column, based on a desired water content. Accordingly, in one embodiment, the invention is to a process for producing acrylic acid comprising the step of withdrawing a purified acetic acid sidedraw from a light ends column in a carbonylation process, wherein a location from which the sidedraw is withdrawn is based on a water content of the sidedraw. The process further comprises the steps of condensing acetic acid of the purified acetic acid stream and alkylenating agent in the presence of a catalyst under conditions effective to form a crude acrylate product comprising acrylic acid and water; and recovering acrylic acid from the crude acrylate product.

In another embodiment, the separation zone 208 may comprise a second column, such as a drying column (not shown). A portion of the purified acetic acid stream 256 may be directed to the second column to separate some of the water from sidedraw 256 as well as other components such as esters and halogens. In these cases, the drying column may yield an acetic acid residue comprising acetic acid and from 0.15 wt. % to 25 wt. % water. The acetic acid residue exiting the second column may be fed to condensation zone 204 in accordance with the present invention.

The purified acetic acid stream, in some embodiments, comprises methyl acetate, e.g., in an amount ranging from 0.01 wt. % to 10 wt. % or from 0.1 wt. % to 5 wt. %. This methyl acetate, in preferred embodiments, may be reduced to form methanol and/or ethanol. In addition to acetic acid, water, and methyl acetate, the purified acetic acid stream may comprise halogens, e.g., methyl iodide, which may be removed from the purified acetic acid stream.

Returning to column 220, low-boiling overhead vapor stream 254 is preferably condensed and directed to an overhead phase separation unit, as shown by overhead receiver decanter 222. Conditions are desirably maintained in the process such that low-boiling overhead vapor stream 254, once in decanter 222, will separate into a light phase and a heavy phase. Generally, low-boiling overhead vapor stream 254 is cooled to a temperature sufficient to condense and separate the condensable methyl iodide, methyl acetate, acetaldehyde and other carbonyl components, and water into two phases. A gaseous portion of stream 254 may include carbon monoxide, and other noncondensable gases such as methyl iodide, carbon dioxide, hydrogen, and the like and is vented from the decanter 222 via stream 260.

Condensed light phase 262 from decanter 222 preferably comprises water, acetic acid, and permanganate reducing compounds (“PRCs”), as well as quantities of methyl iodide and methyl acetate. Condensed heavy phase 264 from decanter 222 will generally comprise methyl iodide, methyl acetate, and PRCs. The condensed heavy liquid phase 264, in some embodiments, may be recirculated, either directly or indirectly, to reactor 214. For example, a portion of condensed heavy liquid phase 264 can be recycled to reactor 214, with a slip stream (not shown), generally a small amount, e.g., from 5 vol. % to 40 vol. %, or from 5 vol. % to 20 vol. %, of the heavy liquid phase being directed to a PRC removal system. This slip stream of heavy liquid phase 264 may be treated individually or may be combined with condensed light liquid phase 242 for further distillation and extraction of carbonyl impurities in accordance with one embodiment of the present invention.

Acetic acid sidedraw 256 from distillation column 220 of carbonylation process 202 is preferably directed to condensation system 204 without further purification. In one embodiment, the acetic acid stream may be a sidestream from a light ends column 220.

In condensation system 204, alkylenating agent feed line 266 and sidedraw 256 comprising acetic acid and water is fed to vaporizer 224, which may be in the form of a single vaporizer or in the form of multiple vaporizers in either parallel or series operations. In one embodiment, alkylenating agent feed in line 266 comprises water. Vapor feed stream 268 is withdrawn and fed to condensation reactor 226. In one embodiment, lines 256 and 266 may be combined and jointly fed to the vaporizer 224. The temperature of vapor feed stream 268 is preferably from 200° C. to 600° C., e.g., from 250° C. to 500° C. or from 340° C. to 425° C. Vapor feed stream 268 comprises from 2 wt. % to 25 wt. % water. For steady state operation, all feeds are vaporized and used in the aldol condensation reaction. In addition, although FIG. 2 shows line 268 being directed to the top of reactor 226, line 268 may be directed to the side, upper portion, or bottom of reactor 226. Further modifications and additional components to reaction zone 204 are described below. In an alternate embodiment, a vaporizer may not be employed and the reactants may be fed directly to reactor 226.

In one embodiment, one or more steam condensate streams from carbonylation system 202 may be used to provide heat for the vaporizer and/or the preheat of the recycle stream. In some embodiments, the one or more steam condensate stream may pre-heat the feed streets prior to them being fed to the vaporizer. As shown in FIG. 2, a portion of steam condensate stream 236 may be used to drive vaporizer 224 through integration with reboiler 272. In some embodiments, steam condensate stream 236′ provides a portion of the energy required to vaporize alkylenating agent and acetic acid in vaporizer 242. In some embodiment, stream 274 exits vaporizer 224 and past through reboiler 272 and return to vaporizer 224. Steam condensate stream 236′ from carbonylation system provides heat for reboiler 272 and reduces the amount of energy necessary from outside sources to vaporizer the alkylenating agent and acetic acid. As a result of the heat transfer, cooled stream 276 may have a lower temperature than steam condensate stream 236′. In one embodiment, temperature of cooled stream 276 is preferably from 110° C. to 200° C., e.g., from 150° C. to 180° C. or from 120° C. to 150° C. In one embodiment, cooled stream 276 exits reboiler 272 and may be returned to carbonylation system 202.

In one embodiment, a portion of steam condensate stream 236 may be used to preheat and/or vaporize the recycle stream. In some embodiments, steam condensate stream 236″ provides a portion of the energy required for the recycle stream to reach reaction temperature. Steam condensate stream 236″ from carbonylation system provides heat for the recycle stream and reduces the amount of energy necessary from outside sources. As a result of the heat transfer, cooled stream 282 may have a lower temperature than steam condensate stream 236″. In one embodiment, temperature of cooled stream 282 is preferably from 110° C. to 200° C., e.g., from 150° C. to 180° C. or from 120° C. to 150° C. In one embodiment, cooled stream 282 exits the heat exchanger or reboiler 278 and may be returned to carbonylation system 202.

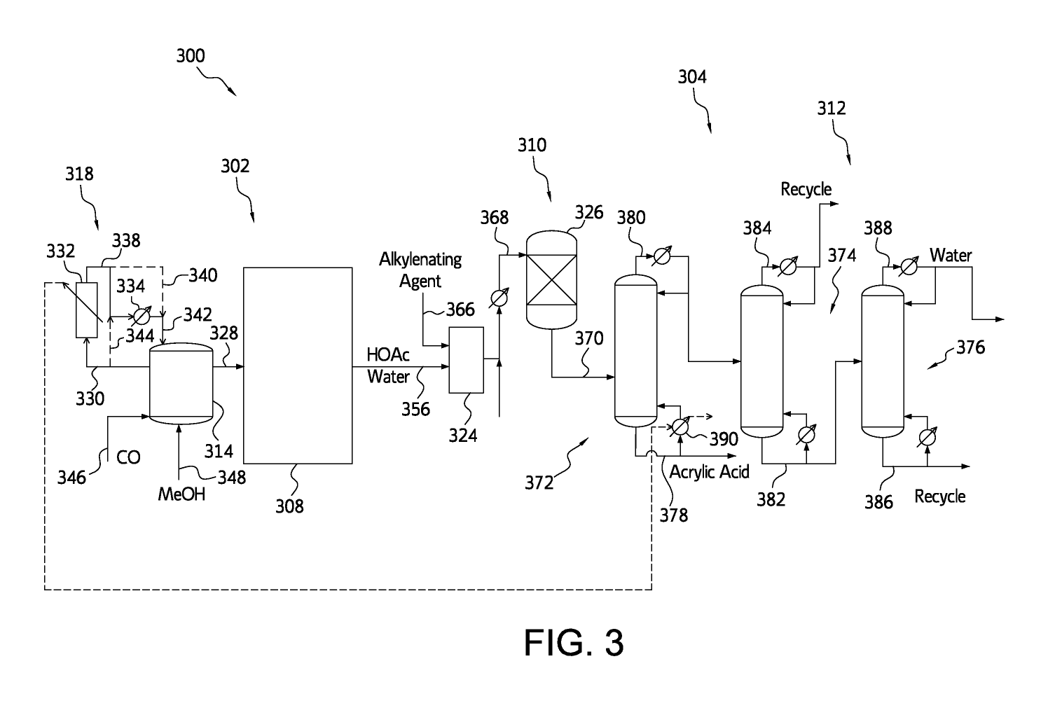

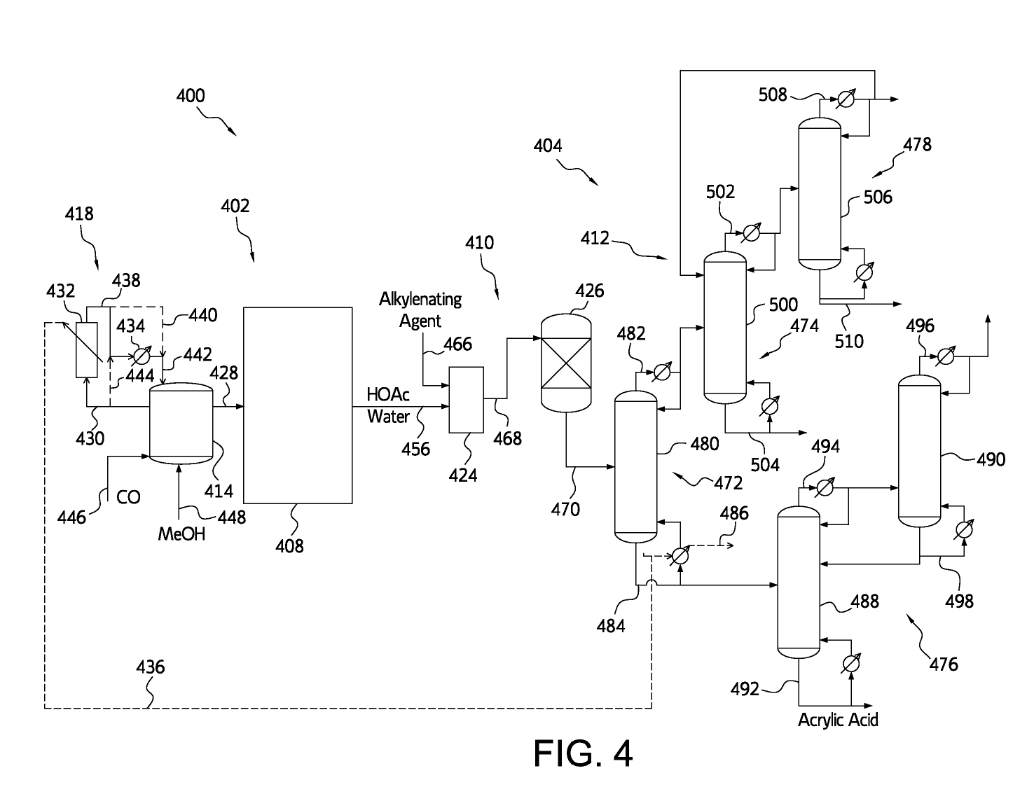

Reactor 226 contains the catalyst that is used in the condensation reaction of the carboxylic acid, preferably acetic acid. During the condensation process, a crude acrylate product is withdrawn, preferably continuously, from reactor 226 via line 270 and directed to acrylate product separation zone 212. Although FIG. 2 shows the crude acrylate product stream being withdrawn from the side of reactor 226, the crude product stream may be withdrawn from any portion of reactor 226. Exemplary composition ranges for the crude product stream are shown in Table 1 above. Crude acrylate stream may be introduced to acrylate product separation zone 212 to yield a purified acrylic acid in line 274, a water stream 284, and a recycle stream 286. Although FIG. 2 shows the steam condensate stream 236 being introduced to vaporizer 224 and preheat recycle stream, steam condensate stream 236 may be introduced to acrylate product separation zone 212, as discussed below in FIGS. 3 and 4.

In one embodiment, recycle stream 286 comprises formaldehyde, water, acetic acid, and acrylic acid. Recycle stream 286 exiting from acrylate production separation zone 212 may be recycled back to reactor 226. In one embodiment, recycle stream 286 may have a lower temperature than the vapor feed stream 268. Recycle stream 286 may past through reboiler 278 and may be combined with vapor feed stream 268.

Acrylic acid may be recovered using a suitable separation scheme, examples of which are discussed herein. FIGS. 3 and 4 illustrate exemplary processes that integrate carbonylation systems and condensation systems. These integrated processes employ various exemplary separation schemes. Of course, other separation schemes (both for the carbonylation system and/or the condensation system) may also be used in accordance with embodiments of the present invention. For purposes of convenience, the columns in each exemplary separation process may be referred to as the first column, second column, third column, etc., but it should be understood that similarly named columns of the embodiments may operate differently from one another.

In FIG. 3, the integration system 300 includes carbonylation systems 302 and condensation system 304. Condensation system 304 includes reaction zones 310 and separation zone 312. Separation zone 312 may optionally comprise a light ends removal unit (not shown) as discussed herein with respect to separation zone 212. As shown in FIG. 3, methanol feed stream in line 348 and carbon monoxide feed stream in line 346 is fed to carbonylation system 302 to yield acetic acid feed stream in line 356. Formaldehyde feed stream in line 366 and acetic acid feed stream in line 356 are fed to vaporizer 324 to create vapor feed stream in line 368, which is directed to reactor 326. In one embodiment, formaldehyde feed stream and acetic acid feed stream may be combined and jointly fed to the vaporizer. Crude acrylate product is withdrawn from the reactor via line 370 and introduced to acrylate product separation zone 312. As stated above in FIG. 2, steam condensate stream 336 may be used to vaporizer acetic acid stream in line 356 and/or formaldehyde feed stream in line 366 and to provide energy to preheat the recycle stream.

As shown in FIG. 3, acrylate product separation zone 312 comprises acrylate product split unit 372, alkylenating agent split unit 374, and acetic acid split unit 376. Acrylate product split unit 372 receives crude acrylic product stream in line 370 and separates them into an acrylate product stream, e.g., stream 378, and an intermediate stream 380 comprising unreacted acetic acid, formaldehyde, and water. At least a portion of the intermediate stream 380 is fed to alkylenating agent split unit 374 to separate them into formaldehyde stream 384 and acetic acid stream 382, which comprises acetic acid and water. Acid stream 382 is fed to acetic acid split unit 376 to separate into acetic acid stream 386 and water stream 388. Formaldehyde from formaldehyde stream 384 and acetic acid from acetic acid stream 382, and optionally from derivatives of acetic acid stream 382, may be returned, directly or indirectly, to vaporizer 324 or reactor 326 to produce additional acrylic acid. In another embodiment, at least a portion of the intermediate stream 380 may be returned, directly or indirectly, to vaporizer 324 or reactor 326 without the complete removal of water. In another embodiment, it may be beneficial to separate the alkylenating agent from the crude acrylate product stream prior to recovering the acrylate product.

Integration system 300 comprises heat transfer system 318, which is similar to heat transfer system 218 as described in FIG. 2. Heat transfer system 318 may generate one or more steam condensate stream 336 using one or more steam generator 343 and/or heat exchanger 334. In one embodiment, reactor solution stream 330 from carbonylation reactor 314 is preferably directed to steam generator 332 to produce steam condensate stream 336 and exiting process stream 338. Exiting process stream 338 may be returned directly to reactor via optional line 340 and return line 342. Reaction solution stream 330 comprises the components of the reaction medium and preferably is retained within the system and not discarded.

Although one column is shown in FIG. 3 for acrylate product split unit 372, alkylenating agent split unit 374 and acetic acid split unit 376, these units may comprise any suitable separation device or combination of separation devices. For example, split units 372, 374, and 376 may comprise at least one column, e.g., a standard distillation column, an extractive distillation column and/or an azeotropic distillation column. In other embodiments, split units 372, 374, and 376 comprise more than one standard distillation columns. In another embodiment, split units 372, 374, and 376 comprise a liquid-liquid extraction unit. Of course, other suitable separation devices may be employed either alone or in combination with the devices mentioned herein.

In operation, as shown in FIG. 3, steam condensate stream 336 is conveyed to condensation system 304, e.g., separation zone 312. In one embodiment, one or more steam condensate streams from carbonylation system 302 may be used to provide heat for one or more distillation columns in separation zone 312. For example, steam condensate stream 336 from carbonylation system may provide heat for reboiler 390 of split unit 372 and may reduce the amount of energy necessary from outside sources for split unit 372. Although steam condensate stream is only shown to be directed to split unit 372, steam condensate stream may be used to provide heat for split units 382 and 386.

As shown in FIG. 3, acrylate product split unit 372 receives at least a portion of crude product stream in line 370 and separates same into acrylic product stream 378 and at least one intermediate stream 380. Acrylate product split unit 372 may yield the finished acrylate product. Intermediate stream 380 may comprise at least 1 wt. % alkylenating agent. As such, intermediate stream 380 may be considered an alkylenating agent stream.

Intermediate stream 380 exiting acrylate product split unit 372 comprises unreacted formaldehyde, acetic acid and water. Exemplary compositional ranges for the streams of acrylate product split unit 372 are shown in Table 2. Components other than those listed in Table 2 may also be present in the streams.

TABLE 2
ACRYLATE PRODUCT SPLIT UNIT

| | Conc. | Conc. | Conc. |
|---|---|---|---|
| | (wt. %) | (wt. %) | (wt. %) |
| Intermediate Stream | | | |
| Acrylic Acid | 0.1 to 40 | 1 to 30 | 0.1 to 30 |
| Acetic Acid | 40 to 99 | 40 to 90 | 50 to 85 |
| Water | 0.1 to 70 | 10 to 60 | 15 to 50 |
| Alkylenating Agent | greater than 1 | 1 to 50 | 1 to 20 |
| Acrylic Product | | | |
| Stream | | | |
| Acrylic Acid | at least 85 | 85 to 99.9 | 95 to 99.5 |
| Acetic Acid | less than 15 | 0.1 to 10 | 0.1 to 5 |
| Water | less than 1 | less than 0.1 | less than 0.01 |
| Alkylenating Agent | less than 1 | 0.001 to 1 | 0.1 to 1 |

In cases where the acrylate product split unit comprises at least one column, the column(s) may be operated at suitable temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 100 kPa, e.g., from 10 kPa to 100 kPa or from 20 kPa to 60 kPa. In preferred embodiments, the pressure at which the column(s) are operated is kept at a low level e.g., less than 50 kPa, less than 27 kPa, or less than 20 kPa. In terms of lower limits, the column(s) may be operated at a pressures of at least 1 kPa, e.g., at least 3 kPa or at least 5 kPa. It has been found that one of the benefits for a low pressure and associated low temperature in the columns of acrylate product split unit 372 is to inhibit and/or eliminate polymerization of the acrylate products, e.g., acrylic acid, which may contribute to fouling of the column(s).

It has also been found that maintaining the temperature of crude acrylate product streams fed to acrylate product split unit 372 at temperatures below 140° C., e.g., below 130° C. or below 115° C., may inhibit and/or eliminate polymerization of acrylate products. In one embodiment, to maintain the liquid temperature under above mentioned temperatures, the pressure of the column(s) is maintained at or below the pressures mentioned above. In these cases, due to restriction of the lower pressures, the number of theoretical column trays is kept at a low level, e.g., less than 10, less than 8, less than 7, or less than 5. As such, multiple columns having fewer trays inhibit and/or eliminate acrylate product polymerization better than a single column having more trays. Specifically, a column having a higher amount of trays, e.g., more than 10 trays or more than 15 trays, would suffer from fouling due to the polymerization of the acrylate products. Thus, in a preferred embodiment, the acrylic acid split is performed in at least two, e.g., at least three, columns, each of which have less than 10 trays, e.g. less than 7 trays. These columns are running in series to achieve the separation targets, but each may operate at the lower pressures discussed above.

In one embodiment (not shown), the acrylate crude product stream is fed to a liquid-liquid extraction column where the acrylate crude product stream is contacted with an extraction agent, e.g., an organic solvent with or without inorganic addition. The liquid-liquid extraction column extracts the acids, e.g., acrylic acid and acetic acid, from the crude product stream. An aqueous stage comprising water, alkylenating agent, and some acetic acid exits the liquid-liquid extraction unit. Small amounts of acrylic acid may also be present in the aqueous stream. The aqueous phase may be further treated and/or recycled. An organic phase comprising acrylic acid, acetic acid, and the extraction agent also exits the liquid-liquid extraction unit. The organic phase may also comprise a small amount of water and formaldehyde. The acrylic acid may be separated from the organic phase and collected as product. The acetic acid may be separated then recycled and/or used elsewhere. The solvent may be recovered and recycled to the liquid-liquid extraction unit.

In one embodiment, depending on the desired purity of the acrylate product, one or more additional distillation column may be used. For example, an additional distillation column (not shown) may be used to separate acrylic product stream 372 to form a final acrylic acid product stream.

In one embodiment, polymerization inhibitors and/or anti-foam agents may be employed in the separation zone, e.g., in the units of the separation zone. The inhibitors may be used to reduce the potential for fouling caused by polymerization of acrylates. The anti-foam agents may be used to reduce potential for foaming in the various streams of the separation zone. The polymerization inhibitors and/or the anti-foam agents may be used at one or more locations in the separation zone.

Returning to FIG. 3, intermediate stream 380 is fed to alkylenating agent split unit 374. As stated above, alkylenating agent split unit 374 may comprise one or more separation devices. Alkylenating agent split unit 374 separates intermediate stream 380 into acid-containing stream 382 and a formaldehyde stream in line 384. Formaldehyde stream 384 may be refluxed and acid-containing stream 382 may be boiled up as shown. Stream 382 comprises at least 1 wt. % acetic acid. As such, stream 382 may be considered an acid stream. Exemplary compositional ranges for the streams of alkylenating agent split unit 374 are shown in Table 3. Components other than those listed in Table 3 may also be present in the residue and distillate.

TABLE 3
ALKYLENATING AGENT SPLIT UNIT

| | Conc. (wt. %) | Conc. (wt. %) | Conc. (wt. %) |
|---|---|---|---|
| Alkylenating | | | |
| Agent Stream | | | |
| Acrylic Acid | 0.1 to 20 | 0.1 to 10 | 0.01 to 5 |
| Acetic Acid | 0.1 to 20 | 0.1 to 10 | 0.01 to 5 |
| Water | 10 to 55 | 15 to 45 | 20 to 40 |
| Alkylenating Agent | at least 1 | 40 to 95 | 50 to 85 |
| Acid Stream | | | |
| Acrylic Acid | <20 | <15 | <10 |
| Acetic Acid | at least 5 | 35 to 99 | 40 to 90 |
| Water | 1 to 50 | 1 to 40 | 1 to 20 |
| Alkylenating Agent | <1 | <0.5 | <0.1 |

In one embodiment, the alkylenating agent stream comprises smaller amounts of acetic acid, e.g., less than 5 wt. %, less than 1 wt. %, or less than 0.1 wt. %. In other embodiments, the alkylenating agent stream comprises higher amounts of alkylenating agent, e.g., greater than 1 wt. % greater than 5 wt. % or greater than 10 wt. %.

In cases where any of the alkylenating agent split unit comprises at least one column, the column(s) may be operated at suitable, but different, temperatures and pressures. For each column, formaldehyde concentration and operating temperature/pressure determine the distribution of formaldehyde in distillate and residue. It is believed that alkylenating agents, e.g., formaldehyde, may be sufficiently volatile under conditions of higher pressures/temperature and lower formaldehyde concentration. Thus, maintenance of the column pressures/temperature at these levels provides efficient formaldehyde separation. In one embodiment, the temperature of the residue exiting the column(s) ranges from 100° C. to 250° C., e.g., from 120° C. to 200° C. or from 150° C. to 200° C. The temperature of the distillate exiting the column(s) preferably ranges from 70° C. to 220° C., e.g., from 90° C. to 170° C. or from 120° C. to 170° C. The pressure at which the column(s) are operated may range from 10 kPa to 2000 kPa, e.g., from 100 kPa to 1500 kPa or from 100 kPa to 1200 kPa. In preferred embodiments, the pressure at which the column(s) are operated is kept at a level greater than 100 kPa, e.g., greater than 500 kPa, or greater than 1000 kPa. In terms of upper limits, the column(s) may be operated at a pressures of less than 6000 kPa, e.g., less than 5000 kPa or less than 4000 kPa. It is believed that above operating conditions will not cause the polymerization of acrylic acid since its concentration has been dropped significantly from acrylate product split unit to alkylenating agent split unit. However, formaldehyde separation can also be conducted at reduced pressure and temperature as discussed below in connection with FIG. 4.

In one embodiment, the alkylenating agent split is achieved via one or more liquid-liquid extraction units. Preferably, the one or more liquid-liquid extraction units employ one or more extraction agents. Multiple liquid-liquid extraction units may be employed to achieve the alkylenating agent split. Any suitable liquid-liquid extraction devices used for multiple equilibrium stage separations may be used. Also, other separation devices, e.g., traditional columns, may be employed in conjunction with the liquid-liquid extraction unit(s).

The inventive process further comprises the step of separating the acid stream to form an acetic acid stream and a water stream. The acetic acid stream comprises a major portion of acetic acid, and the water stream comprises mostly water, e.g., water from the carbonylation reaction, water from formaldehyde feed, and water generated from the condensation reaction. The separation of the acetic from the water may be referred to as dehydration.

As shown in FIG. 3, acid stream 382 exits alkylenating agent split unit 374 and is directed to acetic acid split unit 376 (also known as a drying unit) for further separation, e.g., to remove water from the acetic acid. Acetic acid split unit 376 may comprise any suitable separation device or combination of separation devices. For example, acetic acid split unit 376 may comprise at least one column, e.g., a standard distillation column, an extractive distillation column and/or an azeotropic distillation column. In other embodiments, acetic acid split unit 376 comprises a dryer and/or a molecular sieve unit. In a preferred embodiment, acetic acid split unit 376 comprises a liquid-liquid extraction unit. In one embodiment, acetic acid split unit 376 comprises a standard distillation column as shown in FIG. 3. Of course, other suitable separation devices may be employed either alone or in combination with the devices mentioned herein.

In FIG. 3, acetic acid split unit 376 receives at least a portion of acid stream in line 382 and separates them into a distillate comprising a major portion of water in line 388 and a residue comprising acetic acid and small amounts of water in line 386. The distillate may be refluxed and the residue may be boiled up as shown. In one embodiment, at least a portion of line 386 is returned, either directly or indirectly, to condensation reactor 326.

In another embodiment, at least a portion of the acetic acid-containing stream in either or both of lines 382 and 386 may be directed to an ethanol production system that utilizes the hydrogenation of acetic acid form the ethanol. In another embodiment, at least a portion of the acetic acid-containing stream in either or both of lines 382 and 386 may be directed to a vinyl acetate system that utilizes the reaction of ethylene, acetic acid, and oxygen form the vinyl acetate. In another embodiment, at least a portion of water stream in line 388 is returned to carbonylation system 302.

Exemplary compositional ranges for the distillate and residue of acetic acid split unit are shown in Table 4. Components other than those listed in Table 4 may also be present in the residue and distillate.

TABLE 4
ACETIC ACID SPLIT UNIT

| | Conc. (wt. %) | Conc. (wt. %) | Conc. (wt. %) |
|---|---|---|---|
| Water Stream | | | |
| Acrylic Acid | 0.001 to 10 | 0.001 to 6 | 0.001 to 4 |
| Acetic Acid | 0.001 to 20 | 0.01 to 10 | 0.01 to 6 |
| Water | 80 to 99.9 | 85 to 99.9 | 90 to 99.5 |
| Alkylenating Agent | less than 1 | 0.01 to 5 | 0.01 to 1 |
| Acetic acid stream | | | |
| Acrylic Acid | 0.01 to 30 | 0.01 to 20 | 0.01 to 15 |
| Acetic Acid | 65 to 99.9 | 70 to 99.5 | 75 to 99.5 |
| Water | 0.01 to 15 | 0.01 to 10 | 0.01 to 5 |
| Alkylenating Agent | less than 1 | less than 0.001 | less than 0.0001 |

In cases where the drying unit comprises at least one column, the column(s) may be operated at suitable temperatures and pressures. In one embodiment, the temperature of the residue exiting the column(s) ranges from 80° C. to 250° C., e.g., from 100° C. to 250° C. or from 120° C. to 200° C. The temperature of the distillate exiting the column(s) preferably ranges from 60° C. to 200° C., e.g., from 80° C. to 180° C. or from 100° C. to 160° C. The pressure at which the column(s) are operated may range from 100 kPa to 1000 kPa, e.g., from 100 kPa to 800 kPa or from 300 kPa to 600 kPa.

In some embodiments, a different separation scheme may be used for the recovery of acrylic acid. FIG. 4 illustrates an exemplary separation scheme for the recovery of acrylic acid from the crude acrylate product. As shown in FIG. 4, integrated carbonylation and condensation process 400 comprises carbonylation system 402, which is described above, and condensation system 404. Condensation process includes reaction zone 410 and separation zone 412. Separation zones 412 may optionally comprise a light ends removal unit (not shown) as discussed herein with respect to separation zone 212. As shown in FIG. 4, methanol feed stream in line 448 and carbon monoxide feed stream in line 446 is fed to carbonylation system 402 to yield acetic acid feed stream in line 456. Formaldehyde feed stream in line 466 and acetic acid feed stream in line 456 are fed to vaporizer 424 to create vapor feed stream in line 468, which is directed to reactor 426. In one embodiment, formaldehyde feed stream and acetic acid feed stream may be combined and jointly fed to the respective vaporizer. Crude acrylate product is withdrawn from the reactor via line 470, and introduced to acrylate product separation zone 412.

As shown in FIG. 4, integration system 400 comprises heat transfer system 418, which is similar to heat transfer system 218 and 318, as described in FIGS. 2 and 3. Heat transfer system 418 may generate one or more steam condensate stream 436 using one or more steam generator 432 and/or heat exchanger 434. In one embodiment, reactor solution stream 430 from carbonylation reactor 414 is preferably directed to steam generator 432 to produce steam condensate stream 436 and exiting process stream 438. Exiting process stream 438 may be returned directly to reactor via optional line 440 and return line 442. Reaction solution stream 430 comprises the components of the reaction medium and preferably is retained within the system and not discarded.

As shown in FIG. 4, acrylate product separation zone 412 comprises acrylate agent split unit 472, drying unit 474, acrylate product split unit 476, and methanol removal unit 478. Alkylenating agent split unit 472 may comprise any suitable separation device or combination of separation devices. For example, alkylenating agent split unit 472 may comprise a column, e.g., a standard distillation column, an extractive distillation column and/or an azeotropic distillation column. In other embodiments, alkylenating agent split unit 472 comprises a precipitation unit, e.g., a crystallizer and/or a chiller. Preferably, alkylenating agent split unit 472 comprises a single distillation column.

In another embodiment, the alkylenating agent split is performed by contacting the crude product stream with a solvent that is immiscible with water. For example, alkylenating agent split unit 472 may comprise at least one liquid-liquid extraction column. In another embodiment, the alkylenating agent split is performed via azeotropic distillation, which employs an azeotropic agent. In these cases, the azeotropic agent may be selected from the group consisting of methyl isobutylketene, o-xylene, toluene, benzene, n-hexane, cyclohexane, p-xylene, and mixtures thereof. This listing is not exclusive and is not meant to limit the scope of the invention. In another embodiment, the alkylenating agent split is performed via a combination of distillations, e.g., standard distillation, and crystallization. Of course, other suitable separation devices may be employed either alone or in combination with the devices mentioned herein.

In FIG. 4, alkylenating agent split unit 472 comprises first column 480. The crude product stream in line 470 is directed to first column 480. First column 480 separates the crude product stream to form a distillate in line 482 and a residue in line 484. The distillate may be refluxed and the residue may be boiled up as shown. Stream 482 comprises at least 1 wt % alkylenating agent. As such, stream 482 may be considered an alkylenating agent stream. The first column residue exits first column 480 in line 484 and comprises a significant portion of acrylate product. As such, stream 482 is an intermediate product stream. In one embodiment, at least a portion of stream 482 is directed to drying unit 474.

As shown in FIG. 4, steam condensate stream 436 is conveyed to condensation system 404, e.g., separation zone 412. In one embodiment, one or more steam condensate streams from carbonylation system 402 may be used to provide heat for one or more distillation columns in separation zone 412. For example, steam condensate stream 436 from carbonylation system may provide heat for reboiler 486 of first column 480 and may reduce the amount of energy necessary from outside sources for first column 480. Although steam condensate stream is only shown to be directed to first column 480, steam condensate stream may be used to provide heat for drying unit 474, acrylate product split unit 476, and methanol removal unit 478.

Exemplary compositional ranges for the distillate and residue of first column 480 are shown in Table 5. Components other than those listed in Table 5 may also be present in the residue and distillate.

TABLE 5
FIRST COLUMN

| | Conc. | | |
|---|---|---|---|
| | (wt. %) | Conc. (wt. %) | Conc. (wt. %) |
| Distillate | | | |
| Acrylic Acid | <5 | <3 | <0.1 |
| Acetic Acid | <20 | <10 | 0.01 to 10 |
| Water | >50 | 50 to 90 | 60 to 85 |
| Alkylenating | >5 | 5 to 50 | 10 to 30 |
| Agent | | | |
| Propionic Acid | <1 | <0.1 | <0.01 |
| Methanol | <5 | 0.001 to 5 | 0.01 to 1 |
| Residue | | | |
| Acrylic Acid | 5 to 80 | 10 to 70 | 20 to 60 |
| Acetic Acid | 10 to 80 | 20 to 70 | 30 to 60 |
| Water | <20 | 0.001 to 10 | 0.001 to 1 |
| Alkylenating | <20 | 0.001 to 10 | 0.001 to 1 |
| Agent | | | |
| Propionic Acid | <10 | <8 | <5 |

In one embodiment, the first distillate comprises smaller amounts of acetic acid, e.g., less than 20 wt %, less than 10 wt %, e.g., less than 5 wt % or less than 1 wt %. In one embodiment, the first residue comprises small amounts of alkylenating agent.

In some embodiments, the intermediate acrylate product stream comprises higher amounts of alkylenating agent, e.g., greater than 1 wt % greater than 5 wt % or greater than 10 wt %.

For convenience, the distillate and residue of the first column may also be referred to as the “first distillate” or “first residue.” The distillates or residues of the other columns may also be referred to with similar numeric modifiers (second, third, etc.) in order to distinguish them from one another, but such modifiers should not be construed as requiring any particular separation order.

In one embodiment, polymerization inhibitors and/or anti-foam agents may be employed in the separation zone, e.g., in the units of the separation zone. The inhibitors may be used to reduce the potential for fouling caused by polymerization of acrylates. The anti-foam agents may be used to reduce potential for foaming in the various streams of the separation zone. The polymerization inhibitors and/or the anti-foam agents may be used at one or more locations in the separation zone.

In cases where any of alkylenating agent split unit 472 comprises at least one column, the column(s) may be operated at suitable, but possibly different, temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 10° C. to 90° C., e.g., from 15° C. to 85° C. or from 20° C. to 80° C. The pressure at which the column(s) are operated may range from 1 kPa to 300 kPa, e.g., from 10 kPa to 200 kPa or from 40 kPa to 150 kPa. In preferred embodiments, the pressure at which the column(s) are operated is kept at a low level e.g., less than 300 kPa, less than 200 kPa, or less than 150 kPa. In terms of lower limits, the column(s) may be operated at a pressures of at least 1 kPa, e.g., at least 20 kPa or at least 40 kPa. It is believed that alkylenating agents, e.g., formaldehyde, may be sufficiently volatile at lower pressure at low concentration. In addition, it has been found that, by maintaining a low pressure, as thus reduced temperature, in the columns of alkylenating agent split unit 472 may inhibit and/or eliminate polymerization of the acrylate products, e.g., acrylic acid, which may contribute to fouling of the column(s).

In one embodiment, the alkylenating agent split is achieved via one or more liquid-liquid extraction units. Preferably, the one or more liquid-liquid extraction units employ one or more extraction agents. Multiple liquid-liquid extraction units may be employed to achieve the alkylenating agent split. Any suitable liquid-liquid extraction devices used for multiple equilibrium stage separations may be used. Also, other separation devices, e.g., traditional columns, may be employed in conjunction with the liquid-liquid extraction unit(s).

In one embodiment (not shown), the crude product stream is fed to a liquid-liquid extraction column where the crude product stream is contacted with an extraction agent, e.g., an organic solvent with or without inorganic addition. The liquid-liquid extraction column extracts the acids, e.g., acrylic acid and acetic acid, from the crude product stream. An aqueous phase comprising water, alkylenating agent, and some acetic acid exits the liquid-liquid extraction unit. Small amounts of acrylic acid may also be present in the aqueous stream. The aqueous phase may be further treated and/or recycled. An organic phase comprising acrylic acid, acetic acid, and the extraction agent also exits the liquid-liquid extraction unit. The organic phase may also comprise water and formaldehyde. The acrylic acid may be separated from the organic phase and collected as product. The acetic acid may be separated then recycled and/or used elsewhere. The solvent may be recovered and recycled to the liquid-liquid extraction unit.

The inventive process further comprises the step of separating the intermediate acrylate product stream to form a finished acrylate product stream and a first finished acetic acid stream. The finished acrylate product stream comprises acrylate product(s) and the first finished acetic acid stream comprises acetic acid. The separation of the acrylate products from the intermediate product stream to form the finished acrylate product may be referred to as the “acrylate product split.”

Returning to FIG. 4, intermediate product stream 484 exits alkylenating agent split unit 472 and is directed to acrylate product split unit 476 for further separation, e.g., to further separate the acrylate products therefrom. Acrylate product split unit 476 may comprise any suitable separation device or combination of separation devices. For example, acrylate product split unit 476 may comprise at least one column, e.g., a standard distillation column, an extractive distillation column and/or an azeotropic distillation column. In other embodiments, acrylate product split unit 476 comprises a precipitation unit, e.g., a crystallizer and/or a chiller. Preferably, acrylate product split unit 476 comprises two standard distillation columns as shown in FIG. 4. In another embodiment, acrylate product split unit 476 comprises a liquid-liquid extraction unit. Of course, other suitable separation devices may be employed either alone or in combination with the devices mentioned herein.

In FIG. 4, acrylate product split unit 476 comprises second column 488 and third column 490. Acrylate product split unit 476 receives at least a portion of intermediate acrylic product stream in line 484 and separates same into finished acrylate product stream 492 and at least one acetic acid-containing stream. As such, acrylate product split unit 476 may yield the finished acrylate product.

As shown in FIG. 4, at least a portion of intermediate acrylic product stream in line 484 is directed to second column 488. Second column 488 separates the intermediate acrylic product stream to form second distillate, e.g., line 494, and second residue, which is the finished acrylate product stream, e.g., line 492. The distillate may be refluxed and the residue may be boiled up as shown.

Stream 494 comprises acetic acid and some acrylic acid. The second column residue exits second column 488 in line 492 and comprises a significant portion of acrylate product. As such, stream 492 is a finished product stream. Exemplary compositional ranges for the distillate and residue of second column 488 are shown in Table 6. Components other than those listed in Table 6 may also be present in the residue and distillate.

TABLE 6
SECOND COLUMN

| | Conc. (wt. %) | Conc. (wt. %) | Conc. (wt. %) |
|---|---|---|---|
| Distillate | | | |
| Acrylic Acid | 0.1 to 96 | 1 to 94 | 5 to 92 |
| Acetic Acid | 1 to 95 | 3 to 80 | 5 to 70 |
| Water | <20 | 0.001 to 10 | 0.001 to 1 |
| Alkylenating Agent | <20 | 0.001 to 10 | 0.001 to 1 |
| Propionic Acid | <10 | <8 | <5 |
| Residue | | | |
| Acrylic Acid | 75 to 99.99 | 85 to 99.9 | 95 to 99.5 |
| Acetic Acid | 0.01 to 15 | 0.01 to 10 | 0.08 to 5 |
| Water | <0.001 | <0.01 | <0.05 |
| Alkylenating Agent | <0.001 | <0.01 | <0.05 |
| Propionic Acid | <0.001 | <8 | <5 |

Returning to FIG. 4, at least a portion of stream 494 is directed to third column 490. Third column 490 separates the at least a portion of stream 494 into a distillate in line 496 and a residue in line 498. The distillate may be refluxed and the residue may be boiled up as shown. The distillate comprises a major portion of acetic acid. In one embodiment, at least a portion of line 496 is returned, either directly or indirectly, to reactor 426. The third column residue exits third column 490 in line 498 and comprises acetic acid and some acrylic acid. At least a portion of line 498 may be returned to second column 488 for further separation. In one embodiment, at least a portion of the acetic acid-containing stream in either or both of lines 496 and 498 may be directed to an ethanol production system that utilizes the hydrogenation of acetic acid to form the ethanol. In another embodiment, at least a portion of the acetic acid-containing stream in either or both of lines 496 and 498 may be directed to a vinyl acetate system that utilizes the reaction of ethylene, acetic acid, and oxygen form the vinyl acetate. Exemplary compositional ranges for the distillate and residue of third column 490 are shown in Table 7. Components other than those listed in Table 7 may also be present in the residue and distillate.

TABLE 7
THIRD COLUMN

| | Conc. (wt. %) | Conc. (wt. %) | Conc. (wt. %) |
|---|---|---|---|
| Distillate | | | |
| Acrylic Acid | 0.01 to 50 | 0.1 to 40 | 1 to 35 |
| Acetic Acid | 40 to 99.9 | 50 to 99.5 | 55 to 99 |
| Water | 0.001 to 10 | 0.005 to 5 | 0.01 to 3 |
| Alkylenating Agent | <40 | <25 | <15 |
| Propionic Acid | <10 | <8 | <5 |
| Residue | | | |
| Acrylic Acid | 0.1 to 96 | 1 to 94 | 5 to 92 |
| Acetic Acid | 1 to 95 | 3 to 80 | 5 to 70 |
| Water | <20 | 0.001 to 10 | 0.001 to 1 |
| Alkylenating Agent | <20 | 0.001 to 10 | 0.001 to 1 |
| Propionic Acid | <10 | <8 | <5 |

In cases where the acrylate product split unit comprises at least one column, the column(s) may be operated at suitable temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 110° C., e.g., from 65° C. to 105° C. or from 70° C. to 100° C. The pressure at which the column(s) are operated may range from 1 kPa to 300 kPa, e.g., from 5 kPa to 100 kPa or from 10 kPa to 80 kPa. In preferred embodiments, the pressure at which the column(s) are operated is kept at a low level e.g., less than 300 kPa, less than 100 kPa, or less than 80 kPa. In terms of lower limits, the column(s) may be operated at a pressures of at least 1 kPa, e.g., at least 3 kPa or at least 5 kPa. It has been found that be maintaining a low pressure and thus reduced temperature in the columns of acrylate product split unit 476 may inhibit and/or eliminate polymerization of the acrylate products, e.g., acrylic acid, which may contribute to fouling of the column(s).

Specifically, it has also been found that maintaining the temperature of acrylic acid-containing streams fed to acrylate product split unit 476 at temperatures below 140° C., e.g., below 130° C. or below 115° C., may inhibit and/or eliminate polymerization of acrylate products. In one embodiment, to maintain the liquid temperature at these temperatures, the pressure of the column(s) is maintained at or below the pressures mentioned above. In these cases, due to the restrictions of lower pressures, the number of theoretical column trays is kept at a low level, e.g., less than 10, less than 8, less than 7, or less than 5. As such, it has been found that multiple columns having fewer trays inhibit and/or eliminate acrylate product polymerization better than a single column having more trays. Specifically, a column having a higher amount of trays, e.g., more than 10 trays or more than 15 trays, would suffer from fouling due to the polymerization of the acrylate products. Thus, in a preferred embodiment, the acrylic acid split is performed in at least two, e.g., at least three, columns, each of which have less than 10 trays, e.g. less than 7 trays. These columns each may operate at the lower pressures discussed above.

Returning to FIG. 4, alkylenating agent stream 482 exits alkylenating agent split unit 472 and is directed to drying unit 474 for further separation, e.g., to further separate the water therefrom. The separation of the formaldehyde from the water may be referred to as dehydration. Drying unit 474 may comprise any suitable separation device or combination of separation devices. For example, drying unit 474 may comprise at least one column, e.g., a standard distillation column, an extractive distillation column and/or an azeotropic distillation column. In other embodiments, drying unit 474 comprises a dryer and/or a molecular sieve unit. In a preferred embodiment, drying unit 474 comprises a liquid-liquid extraction unit. In one embodiment, drying unit 474 comprises a standard distillation column as shown in FIG. 4. Of course, other suitable separation devices may be employed either alone or in combination with the devices mentioned herein.

In FIG. 4, drying unit 474 comprises fourth column 500. Drying unit 474 receives at least a portion of alkylenating agent stream in line 482 and separates them into a fourth distillate comprising water, formaldehyde, and methanol in line 502 and a fourth residue comprising mostly formaldehyde and water in line 504. The distillate may be refluxed and the residue may be boiled up as shown. In one embodiment, at least a portion of line 504 is returned, either directly or indirectly, to reactor 426.

Exemplary compositional ranges for the distillate and residue of fourth column 500 are shown in Table 8. Components other than those listed in Table 8 may also be present in the residue and distillate.

TABLE 8
FOURTH COLUMN

| | Conc. (wt. %) | Conc. (wt. %) | Conc. (wt. %) |
|---|---|---|---|
| Distillate | | | |
| Acrylic Acid | <0.1 | 0.0001 to 0.5 | 0.001 to 0.1 |
| Acetic Acid | <10 | 0.001 to 5 | 0.01 to 3 |
| Water | 25 to 99 | 35 to 97 | 45 to 95 |
| Alkylenating Agent | 1 to 30 | 3 to 25 | 5 to 20 |
| Methanol | <3 | 0.01 to 3 | 0.1 to 2 |
| Residue | | | |
| Acrylic Acid | <5 | 0.01 to 3 | 0.01 to 1 |
| Acetic Acid | 0.001 to 10 | 0.01 to 8 | 0.1 to 5 |
| Water | 1 to 50 | 5 to 45 | 10 to 40 |
| Alkylenating Agent | 1 to 80 | 10 to 75 | 20 to 70 |
| Propionic Acid | <10 | <8 | <5 |

In cases where the drying unit comprises at least one column, the column(s) may be operated at suitable, but possibly different, temperatures and pressures. In one embodiment, the temperature of the residue exiting the column(s) ranges from 60° C. to 120° C., e.g., from 65° C. to 110° C. or from 70° C. to 100° C. The temperature of the distillate exiting the column(s) preferably ranges from 20° C. to 90° C., e.g., from 25° C. to 80° C. or from 30° C. to 60° C. The pressure at which the column(s) are operated may range from 1 kPa to 500 kPa, e.g., from 5 kPa to 300 kPa or from 10 kPa to 100 kPa.

Returning to FIG. 4, alkylenating agent stream 502 exits drying unit 500 and is directed to methanol removal unit 478 for further separation, e.g., to further separate the methanol therefrom. Methanol removal unit 478 may comprise any suitable separation device or combination of separation devices. For example, methanol removal unit 478 may comprise at least one column, e.g., a standard distillation column, an extractive distillation column and/or an azeotropic distillation column. In one embodiment, methanol removal unit 478 comprises a liquid-liquid extraction unit. In a preferred embodiment, methanol removal unit 478 comprises a standard distillation column as shown in FIG. 4. Of course, other suitable separation devices may be employed either alone or in combination with the devices mentioned herein.

In FIG. 4, methanol removal unit 478 comprises fifth column 506. Methanol removal unit 506 receives at least a portion of line 502 and separates them into a fifth distillate comprising methanol and formaldehyde in line 508 and a fifth residue comprising water and formaldehyde in line 510. The distillate may be refluxed and the residue may be boiled up (not shown). In one embodiment, at least a portion of line 508 is returned to drying column 500 to recover formaldehyde and at least another portion is directed out of the condensation system in order to keep methanol balance. The latter portion containing methanol may return to carbonylation reactor 414, to formaldehyde system, to furnace, or to other locations

Exemplary compositional ranges for the distillate and residue of fifth column 506 are shown in Table 9. Components other than those listed in Table 9 may also be present in the residue and distillate.

TABLE 9
FIFTH COLUMN

| | Conc. (wt. %) | Conc. (wt. %) | Conc. (wt. %) |
|---|---|---|---|
| Distillate | | | |
| Acrylic Acid | <0.1 | <0.01 | <0.001 |
| Acetic Acid | <0.1 | <0.01 | <0.001 |
| Water | 30 to 90 | 40 to 85 | 50 to 80 |
| Alkylenating Agent | 1 to 45 | 10 to 40 | 15 to 35 |
| Methanol | <20 | <10 | <5 |
| Residue | | | |
| Acrylic Acid | <0.01 | <0.005 | <0.001 |
| Acetic Acid | 0.01 to 10 | 0.01 to 5 | 0.01 to 3 |
| Water | 70 to 99.9 | 80 to 99.7 | 85 to 99.5 |
| Alkylenating Agent | <0.01 | <0.005 | <0.1 |
| Methanol | <0.1 | <0.05 | <0.01 |

In cases where the methanol removal unit comprises at least one column, the column(s) may be operated at suitable, but possibly different, temperatures and pressures. In one embodiment, the temperature of the residue exiting the column(s) ranges from 100° C. to 200° C., e.g., from 110° C. to 190° C. or from 120° C. to 180° C. The temperature of the distillate exiting the column(s) preferably ranges from 80° C. to 180° C., e.g., from 90° C. to 180° C. or from 100° C. to 170° C. The pressure at which the column(s) are operated may range from 100 kPa to 1000 kPa, e.g., from 150 kPa to 900 kPa or from 200 kPa to 800 kPa.

### EXAMPLE

### Example 1

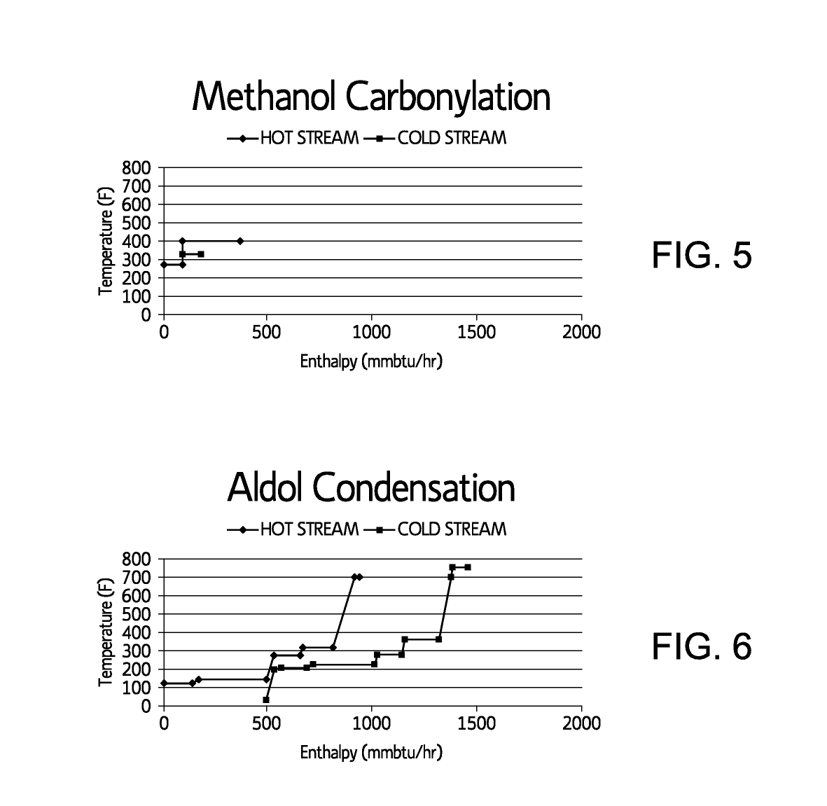

FIGS. 5 and 6 are charts showing the composite curves of utility requirements for the hot and cold streams for the carbonylation process and the aldol condensation reaction process, respectively. FIG. 5 shows the composite curve of utility requirements for the production of 1,200 kTa of acetic acid product. FIG. 6 shows the composite curve of utility requirements for the production of 200 kTa of acrylic acid. It has been measured that the net energy requirement for the carbonylation process is −187.43 mmbtu/hr and the net energy requirement for the aldol condensation reaction process is calculated to be 522.47 mmbtu/hr. Therefore, the net energy requirement for these two processes without integration is 335.04 mmbtu/hr.

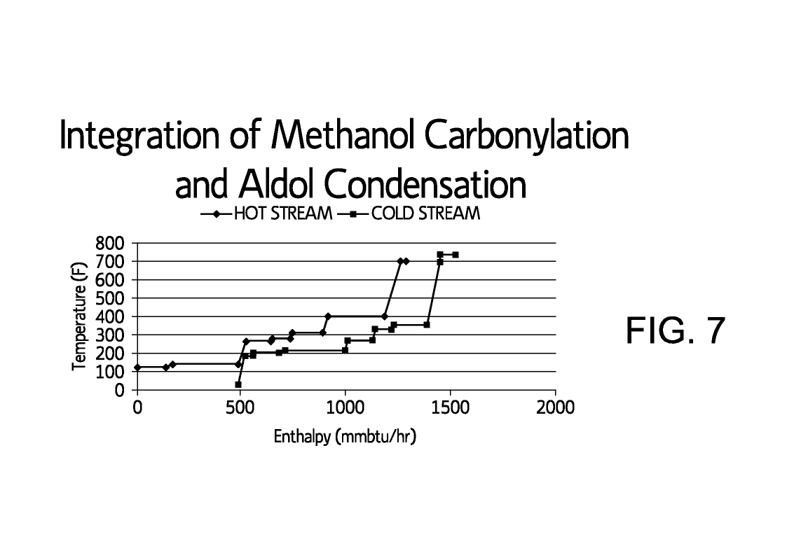

FIG. 7 is a chart showing the composite curve of utility requirement for the hot and cold streams for the integrated carbonylation and aldol condensation reaction processes as shown in FIG. 3. It has been calculated that the net energy requirement for the integrated process is 247.8 mmbtu/hr. Therefore, the integration of the two processes beneficially reduces the energy requires to produce acrylic acid. Above analysis can be extended to different capacity combinations of acetic acid and acrylic acid and similar energy benefits can be reached by heat integration of these two systems.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for producing an acrylate product, comprising:
reacting, in a carbonylation system, carbon monoxide with at least one reactant in a reaction medium under conditions effective to produce a crude alkanoic acid stream, wherein the reaction is an exothermic carbonylation reaction and wherein the reaction medium is a heterogeneous system with solid catalyst or a homogeneous system comprising water, methyl iodide, and a first homogeneous catalyst;
separating the crude alkanoic acid stream to form an alkanoic acid product stream comprising alkanoic acid and water;
removing from the carbonylation system at least a portion of heat generated by the carbonylation reaction;
transferring at least a portion of the heat generated by the carbonylation reaction to a heat transfer system that utilizes at least one steam condensate stream to convey the generated heat;
contacting, in a condensation reaction zone, at least a portion of the alkanoic acid in the alkanoic acid product stream with an alkylenating agent in a reactor in the presence of a second catalyst under conditions effective to form a crude acrylate product stream comprising the acrylate product;
conveying at least a portion of the at least one steam condensate stream to the condensation reaction zone; and
separating, in a condensation separation zone, the crude acrylate product stream to form an acrylate product stream and a water stream.

2. The process of claim 1, wherein the conveying comprises conveying a portion of the heat of the at least one steam condensate stream to a vaporizer.

3. The process of claim 1, further comprising vaporizing the alkanoic acid product stream and the alkylenating agent in the condensation reaction zone using the at least one steam condensate stream.

4. The process of claim 1, wherein the at least one steam condensate stream has a temperature of at least 130° C.

5. The process of claim 1, wherein the conveying comprises conveying a portion of the heat of the at least one steam condensate stream to the reactor.

6. The process of claim 1, wherein the conveying comprises conveying a portion of the heat of the at least one steam condensate stream to the condensation separation zone.

7. The process of claim 1, further comprising returning at least a portion of the at least one steam condensate stream to the carbonylation system.

8. The process of claim 1, wherein the condensation separation zone comprises at least one separation unit and at least a portion of the at least one steam condensate stream provides heat to the at least one separation unit.

9. The process of claim 8, wherein the at least one steam condensate stream is conveyed to a reboiled stream of the at least one column of the condensation separation zone.

10. The process of claim 1, wherein the separating comprises separating the crude alkanoic acid in a light ends column.

11. The process of claim 10, further comprising withdrawing the alkanoic acid product stream as a sidedraw from the light ends column.

12. The process of claim 1, wherein the alkanoic acid product stream comprises from 0.5 wt. % to 25 wt. % water.

13. The process of claim 1, wherein the separating comprises separating at least a portion of the crude acrylate product stream to form an alkylenating agent stream comprising at least 1 wt. % alkylenating agent and a product stream comprising at least 10 wt. % acrylate product.

14. The process of claim 1, wherein the crude acrylate product stream comprises at least 1 wt % alkylenating agent.

15. The process of claim 1, the crude acrylate product stream comprises:
at least 10 wt. % acrylate product; and
at least 1 wt. % alkylenating agent.
